(19) United States
(12) Patent Application Publication
(10) Pub. No.: US 2011/0294683 A1
(43) Pub. Date: Dec. 1, 2011

(54) BIOMARKERS

(75) Inventors: Yvan Devaux, Zoufftgen (FR); Daniel R. Wagner, Bertange (LU); Francisco Azuaje, Strassen (LU); Mélanie Vausort, Strassen (LU)

(73) Assignee: Centre de Recherche Public de la Sant'e, Strassen (LU)

(21) Appl. No.: 12/998,543

(22) PCT Filed: Oct. 30, 2009
(86) PCT No.: PCT/EP2009/064410

§ 371 Date: Aug. 4, 2011

Related U.S. Application Data
(60) Provisional application No. 61/109,649, filed on Oct. 30, 2008.

(51) Int. Cl. C40B 30/04; G06F 17/30; G06F 19/00; C12Q 1/68; G01N 33/566

(52) U.S. Cl. 506/9; 435/6.12; 435/7.92; 435/7.94; 702/19; 707/792; 707/769; 707/E17.014

(57) ABSTRACT

Provided is a method of identifying myocardially-infarcted patients having an increased risk of developing a heart condition.

### FIELD OF THE INVENTION

The present invention relates to a set of new biomarkers for assessing the risk or severity of Heart Failure (HF) or ventricular remodeling in a patient, particularly after the patient has suffered from a myocardial infarction (MI), and diagnostic kits to measure levels of these biomarkers.

### BACKGROUND OF THE INVENTION

Heart failure (HF) is not a specific disease, but a compilation of signs and symptoms, all of which are caused by an inability of the heart to appropriately increase cardiac output as needed. Patients typically present with shortness of breath, edema and fatigue. HF has become a disease of epidemic proportion, affecting 3% of the adult population. Mortality of HF is worse than many forms of cancer with a five-year survival of less than 30%. Myocardial infarction (MI) is one of the leading causes of HF. 63% of the patients develop HF in the 6 years following MI. Left ventricular remodeling contributes largely to HF. Because HF becomes more common in the elderly, the number of affected individuals will continue to rise with our ageing population.

Extensive research in the last decade has brought a better understanding of the pathophysiology of HF. At the same time, many new therapeutic targets have been identified, although only a few of them are of potential therapeutic use. Some of these targets have been called “prognostic biomarkers” or “biomarkers” when their prognostic value could be demonstrated.

Biomarkers can be classified into three categories. Biomarkers that can assist in the care of apparently healthy individuals are called “screening biomarkers.” Biomarkers seen in patients having a suspicion of disease are called “diagnostic biomarkers,” and biomarkers seen in patients with overt disease are called “prognostic biomarkers.” While diagnostic biomarkers such as troponin I and troponin T for MI and brain natriuretic peptide (BNP) for heart failure are used in clinical practice, the potential use of these biomarkers as prognostic biomarkers to tailor the treatment to the individual patient (“personalized medicine”) has yet to be proved.

It has to be noted that the earlier the identification of a patient prone to develop HF after MI can be made, the more efficiently the treatment can be adjusted. However, a major limitation of the use of individual biomarkers is that not all patients may present such risk factors independently. Therefore, it has become evident that the early identification of patients prone to develop HF after MI would considerably benefit from the multiplication and integration of biomarkers.

Vascular endothelial growth factor (VEGF), a sub-family of growth factors, is a broad term covering a number of proteins from a number of families. These growth factors have mostly been studied for their angiogenic properties. Various growth factors have been identified to date, the most well known of which is VEGFA. VEGFA is often simply referred to as “VEGF” (Ferrara et al. “The biology of VEGF and its receptors” Nat Med. 2003 jun; 9(6):669-676). One of the proposed biomarkers of the present invention, VEGFB, is part of the family of vascular endothelial growth factors but is distinct from VEGFA.

### OBJECTS OF THE INVENTION

The objects of the present invention are:

### 1. To Provide a Prognostic Tool

It is an object of the present invention to provide a tool for early prognosis of the occurrence of HF in order to improve survival and to lessen the development of worsening HF.

It is another object of the present invention to use this prognostic tool to identify patients at risk to develop ventricular remodeling and heart failure.

It is another object of the present invention to use this prognostic tool to adjust treatments to better prevent the development of ventricular remodeling and HF after MI.

### 2. To Provide a New Diagnostic Kit

It is an object of the present invention to provide a diagnostic kit able to measure marker concentrations in biological fluids in order to help in the early prognosis of the occurrence of HF to improve survival and to lessen the development of worsening HF.

It is another object of the present invention to provide a new diagnostic kit to identify patients at risk to develop ventricular remodeling and HF.

It is another object of the present invention to provide a new diagnostic kit to adjust treatments to better prevent the development of ventricular remodeling and HF after MI.

Furthermore, it is also an object of the present invention to provide biomarkers that can be used in screening patients, post-MI, for the susceptibility of a patient to develop HF or Ventricular Remodeling.

Surprisingly, we have found that the mRNA and plasma levels of the following proteins VEGFB, THBS1 and PGF vary post MI and are excellent indica of the likelihood of the patient to go on to develop HF and/or Ventricular Remodeling.

These biomarkers can, therefore, be used to screen MI patients and, in particular, provide an early prognostic tool for identifying those patients who, having suffered from MI, are at an increased risk of then going on to develop HF and/or Ventricular Remodeling. Diagnostic kits for measuring the levels of these three biomarkers are also provided and are useful in the context of MI to predict the occurrence of HF and/or Ventricular Remodeling.

### SUMMARY OF THE INVENTION

According to a first aspect, the present invention provides a method of identifying myocardially-infarcted patients having an increased risk of developing a heart condition, comprising:
assaying, post infarction, a body fluid sample from the patient for levels of Vascular

Endothelial Growth Factor B (VEGFB), Thrombospondin-1 (THBS1) and/or Placental Growth Factor (PGF);
comparing the levels VEGFB, THBS1 and/or PGF with the corresponding levels of VEGFB, THBS1 and/or PGF from a reference sample, said reference sample having a known clinical outcome; and
determining whether the patient has an increased risk of developing a heart condition, based on said comparison.

Also provided, is a method of identifying myocardially-infarcted patients having an increased risk of developing a heart condition, comprising:
assaying, post infarction, a body fluid sample from the patient for levels of Vascular Endothelial Growth Factor B (VEGFB), Thrombospondin-1 (THBS1) and/or Placental Growth Factor (PGF);
comparing the levels of VEGFB, THBS1 and/or PGF with the corresponding levels of VEGFB, THBS1 and/or PGF with a control; and
determining whether the patient has an increased risk of developing a heart condition, based on said comparison.

In some embodiments, at least one of the following indicates an increased likelihood of said patient suffering from said heart condition:
lower levels of VEGFB in the assayed patient sample compared to the VEGFB control level;
higher levels of THBS1 in the assayed patient sample compared to the THBS1 control level; and/or
higher levels of PGF in the assayed patient sample compared to the PGF control level.

Preferably, the heart condition may be myocardial infarction, acute coronary syndrome, ischemic cardiomyopathy or non-ischemic cardiomyopathy. More preferably, the patient may go on to develop or suffer from heart failure. Preferably, the patient may undergo ventricular remodeling. It will be appreciated that many myocardially-infarcted patients undergo ventricular remodeling and subsequently, or at the same time, develop the condition known as heart failure. Therefore, there is a clear correlation between ventricular remodeling and heart failure and, preferably, myocardially infarcted patients undergoing ventricular remodeling will also develop heart failure.

It is also preferred that the body fluid sample taken from the patient is a blood sample, a tissue fluid sample, a plasma sample, a serum sample or a urine sample.

Preferably, the levels of VEGFB, THBS1 and/or PGF assayed are mRNA levels. These may be determined by assaying mRNA in red and/or white blood cells. Preferably, the blood cells are leukocytes, neutrophils, basophils, eosinophils, lymphocytes, monocytes, platelets, or erythrocytes.

Preferably, the VEGFB, THBS1 and/or PGF may be measured at the mRNA level from blood cells by any technique able to quantitate mRNA, most preferably quantitative PCR, most preferably microarrays. It is also preferred that, the VEGFB, THBS1 and/or PGF may be measured at the protein level in the plasma by any technique able to quantitate proteins, most preferably ELISA. mRNA assay results may be used in combination with plasma protein assay results for a more accurate assessment.

Preferably, only the level of PGF is assayed, which may preferably be any of the sequences for PGF given in SEQ ID NOS 7-9, or fragments thereof. It is also preferred that only the level of THBS1 is assayed, which may preferably be an any of the sequences for THBS1 given in SEQ ID NOS 4-6, or fragments thereof. More preferably, however, both the levels of THBS1 and PGF are assayed.

It is particularly preferred that the level of VEGFB is assayed, which may preferably be any of the sequences for VEGFB given in SEQ ID NOS 1-3, or fragments thereof. This may be alone or in combination with THBS1 and/or PGF. It is preferred that the VEGFB may be either the splicing variant VEGFB 186, the splicing variant VEGFB 167 or both.

Preferably, the mRNA level is assayed the day of myocardial infarction. In other preferred embodiments, mRNA or plasma protein samples for assaying are obtained from the patient on day 1, being the day following myocardial infarction. Preferably, a subsequent sample, obtained from the patient later on day 1 or on day 2 or 3 or 4 or 5 or 6 or 7 or at any time up to 1 month post infarction, is assayed.

Preferably, the subsequent sample is obtained from the patient on day 1 and the assayed value from day 1 is compared with the reference sample. However, it is preferred that the subsequent sample is obtained from the patient on day 1 and the assayed value from day 1 is compared with the assayed levels in the sample from the day of infarction. This is most preferable in respect of VEGFB. In other words, it is especially preferred that the control, first post-MI and subsequent samples are assayed for levels of VEGFB and that these VEGFB levels are compared. An increase in VEGFB levels from the reference sample to day 1 or 2 is particularly useful in identifying the patient as being at lower risk of developing said condition. Similarly, a decrease in VEGFB levels from the control sample to day 1 or 2 is particularly useful in identifying the patient as being at a higher risk of developing said condition.

However, it is also preferred that the VEGFB levels from the first post-MI sample at day 0 or 1 to the subsequent sample (post MI) at day 1 or 2 are compared, as an increase in VEGFB from day 0 or 1 to day 1 or 2 is highly indicative of a patient with lowered risk of developing the present conditions. Changes in VEGFB measured between day 0 and day 2 or day 1 and day 3 are particularly preferred.

Indeed, we found that plasma VEGFB levels were similar between high and low EF groups at day 0 and day 1 after MI. At day 2 however, VEGFB levels increased in high EF patients (2 fold compared with day 0) whereas they dropped in low EF patients (2.5 fold compared with day 0) (FIG. 9). These data are in accordance with the up regulation of VEGFB mRNA in high EF patients (FIG. 8). These results suggest that patients able to increase their production of VEGFB after MI are more prone to have a favourable outcome.

A decrease in VEGFB from day 0 or 1 to day 1 or 2, and in particular day 0 to 2, is highly indicative of a patient with an elevated risk of developing the present conditions.

The present invention measures changes in the levels of expression or prevalence of certain biomarkers, rather than just the presence or absence thereof, as is sometimes the case in the art. In order to measure a change in said levels, a point of reference needs to be established. This can preferably be the levels in a further sample, in particular an earlier sample, preferably taken on the day of infarction (day zero) or on the first or second day following infarction. Preferably, the control sample is the basal level of VEGFB, THBS1 or PGF on the day of infarction, respectively.

Alternatively, the control can be a reference value obtainable from a population of infracted patients with a known range of clinical outcomes. A database can be built up of data from infarcted patients and once calibrated for age, sex etc, an average (mode, mean or median as deemed appropriate) value or value range can be ascertained for patients having certain criteria (for instance sex, weight and age), measured at a particular time post infarction, and with known clinical outcomes (i.e. heart failure or not). The data from the assayed patient can then be compared against this reference value or range to determine the likelihood of the assayed patient having one or other of the clinical outcomes. For instance, if it established that VEGFB levels lower than around “X” micrograms\ml in the plasma of infarcted male patients aged 55-60 measured on day 1 post infarction gives a 50% likelihood of heart failure, then an assayed sample can be taken from a corresponding male on day 1 and compared to this value to determine if the 50% likelihood applies to this patient.

The determination step may be by suitable statistical analysis, for instance “nearest neighbor” comparison techniques, such as the Kstar and SVM programs. A particularly preferred example is to use a data mining platform, such as Weka. This may be followed by hierarchical clustering, preferably implemented using unweighted pair-group method with arithmetic averages and correlation coefficients. Clustering visualization may then be performed with GEPAS. Optionally, statistical significance tests, Pearson correlation values, and graphical plots may be generated with the Statistica package (v. 6.0).

The method may further comprise collecting data on one or more MI patients, the data preferably to include the levels or values of at least one of, and preferably each of, the three mRNAs and/or plasma proteins (VEGFB, THBS1 and/or PGF) and the associated clinical outcome for that patient. This is used to create feature/value data for VEGFB, THBS1 and/or PGF associated with a particular clinical outcome.

Thereafter, a database may be populated with the data from each individual. These known values could be referred to as reference (or “seen”) samples.

“Query” or “unseen” sample data, i.e. from a recently infarcted new patient, is inputted and queried against the reference data in the database. The query data is obtained from a sample collected from a patient who has just had an MI and for whom it is desired to establish the likelihood of developing Heart Failure. In other words, the clinical outcome for this patient is unknown and the operator is asking the program to predict this patient's outcome (increased or reduced risk of HF post-MI).

The program compares the unseen/query sample data with the reference data set and makes its prediction on the clinical outcome of the patient.

Where reference is made to mRNA above, it will be appreciated that the same holds true for the plasma protein levels.

A classifier is preferred to determine a prognosis. The classifier may include programs such as PAM, Kstar and SVM which are well known to make a prediction in different ways. However, there is still always a comparison of the mRNA (or protein) data in the “unseen” sample with one or more of the “seen” data points, for instance when searching for the “nearest neighbor.” Kstar and SVM, for instance, use different algorithms, but essentially work in similar ways by comparing the query data or values against the nearest reference set of values.

It is, therefore, preferred that the classifier searches the database and compares the query/unseen data with the known/seen data. Having found the “closest match” (for instance in a 3-D sense when analyzing all three mRNA or plasma protein levels) in the database, the program bases its prediction for the clinical outcome of the query patient based on the clinical outcome of that closet match.

Also provided is a method comprising:
analyzing a body fluid sample from a post-MI patient for mRNA or plasma protein levels of VEGFB, THBS1 and/or PGF to determine feature data of the patient, associating this feature data with a particular clinical outcome relating to the incidence of Heart Failure in said patient, and entering this feature data into a database;
repeating the analysis for a plurality of post-MI patients, to populate a database to contain reference information about a relationship between incidence of HF and levels of VEGFB, THBS1 and/or PGF;
determining feature data of a post-MI patient with an unknown prognosis; and
processing the feature data for the VEGFB, THBS1 and/or PGF levels from the patient to compare it with the feature data in the database; and
outputting a likely prognosis for the post-MI unknown prognosis patient in dependence upon the result of the comparison.

These steps may also be included in the method according to the first aspect of the invention.

The likely prognosis may be an increased or reduced risk of HF, which can then affect the clinician's further proscribed treatment for the patient. The terms prognosis and diagnosis may be used interchangeably, unless otherwise apparent.

Also provided is a database comprising feature data from MI patients, the feature data including the clinical outcome of the patient matched to at least one of, and preferably all three of, VEGFB, THBS1 and PGF levels assayed post-MI, as described herein. The invention also provides a comparing device such as a computer for accessing the database and/or processing the query. The invention also provides a system comprising a database and at least one computer to access and/or operate the database. A or the computer may also be used to process or administer the querying of the database with the feature data of the patient to be tested.

The database may be stored centrally, for instance in a server, or may be retained in the lab or field equipment used to assay the levels of VEGFB, THBS1 and/or PGF, or in a computer associated with said equipment, as discussed below. The computer may also be located centrally with the server or remotely, for instance in an intermediate lab, or located in field equipment.

A kit, which may include lab or field equipment for assaying a sample from the patient is also provided. The equipment may comprise the database or may simply comprise a display or readout of the results of the analysis. The equipment may have the ability to contact the database remotely, for instance via an internet network or the internet, whether by wire or by wireless broadcast.

The invention also provides a method of obtaining feature data from a patient, by assaying VEGFB, THBS1 and/or PGF levels and preferably associating the level data with a patient, for instance by using a patient identifier, such as a code.

Also provided is a method of receiving feature data for the VEGFB, THBS1 and/or PGF levels from the patient to compare it with the feature data in the database. The data may be processed by a receiver or transmitted to a processor.

Thus, the invention further provides a method of processing feature data for the VEGFB, THBS1 and/or PGF levels from the patient and comparing it with the feature data in the database. The likely prognosis for the patient may be outputted by the processor in dependence upon the result of the comparison and may, optionally, be transmitted to a separate computer and/or the kit or equipment discussed above, via a network, the internet and by wire or wireless transmission.

The database may also be stored on a carrier medium, such as a disk or memory device. Thus, the invention also provides a carrier medium comprising a database arranged to cause a computer to determine the likelihood of a patient developing HF post-MI when queried with feature data on VEGFB, THBS1 and/or PGF levels from a patient.

The levels of VEGFB, THBS1 and/or PGF may be considered to be values or ratios and do not necessarily have to be volumes or mass per unit volume and so forth.

Preferably, the reference sample is from a patient in a similar demographic, genotypic or phenotypic group to the patient. The reference sample may be considered to be in the same demographic group as the infarcted patient if any number of the following criteria are met: sex, age, race or ethnic background, and medical history. Suitable genotypic or phenotypic control samples can be selected based on any number of suitable selection criteria, such as determining the genotype of a patient at one or more loci, in particular those known to be associated with infarcted patients, heart failure and/or ventricular remodeling.

Determining the genotype may comprise detecting the presence of an amino acid change in the sequence of the hemopexin domain of MMP-9 (Matrix Metalloproteinase 9), the presence of an amino acid change in said domain being indicative of susceptibility to said heart condition, post myocardial infarction. Preferably, the sequence that is detected comprises or encodes either a Glutamine (Gln) or an Arginine (Arg) amino acid residue at a position corresponding to position 148 of the hemopexin domain of MMP-9. Preferably, the detected sequence is SEQ ID NO. 13, which is the amino acid sequence of the hemopexin domain of MMP-9 from the at risk (susceptible) group (showing Arg at amino acid position 148), or a polynucleotide sequence encoding it. The Single Nucleotide Polymorphism (SNP), present in the coding sequence of the MMP-9 gene, is found at different frequencies in patients with good or poor prognoses for heart failure following myocardial infarction and can lead to a single amino acid change in the hemopexin domain of the transcribed and active MMP-9 protein, resulting in an electrostatic change in the site on MMP-9 that binds tissue inhibitor of metalloproteinase-1 (TIMP-1). This is significant as TIMP-1 is the foremost inhibitor of MMP-9 activity. Thus, it may be useful to determine whether the present patient has the above SNP and then, if he does, compare the levels of VEGFB, THBS1 and/or PGF (in the assayed samples of the present invention) with the levels in samples from patients also known to have the above SNP, or vice versa.

Preferably, the determination of a decreased risk of a heart condition is relative to those infarcted reference patients having relatively high levels of VEGFB mRNA (>−1.4), relatively low levels of THBS1 (<0) and/or relatively low levels of PGF (<−0.1) respectively. These values are expressed as log ratio (patient RNA/reference RNA). The reverse holds true for an increased risk of developing a heart condition.

The above values are derived from Table 4, although it is preferred that these may vary by at least 1 or 2%, more preferably at least 5%, more preferably at least 7%, more preferably at least 10%, more preferably at least 15%, more preferably at least 20%, more preferably at least 25%, more preferably at least 30%, more preferably at least 40%, and even up to 50%.

Since the only established biomarker of HF (Heart Failure) is pro-BNP (brain natriuretic peptide), the prognostic performance of the 3 biomarkers set disclosed here was compared with the prognostic performance of NT-pro-BNP. The prognostic performance of the plasma level of NT-pro-BNP, measured 1 day after MI, was moderate (AUC=0.63, Table 5). Therefore the set of 3 biomarkers disclosed here clearly outperformed the prognostic value of NT-pro-BNP. Nevertheless, it is preferred that BNP can also be assayed in the present method, either to increase the accuracy or confirm a determined prognosis. This BNP assayed level may be compared to a BNP basal or a reference level as discussed herein.

The nucleotide and protein sequences for pro-BNP are provided in SEQ ID Nos 10-11.

Also provided is a method of establishing a prognosis for a myocardially-infarcted patient, the method comprising:
assaying, post infarction, a body fluid sample from the patient for levels of Vascular Endothelial Growth Factor B (VEGFB) and levels of Thrombospondin-1 (THBS1) and/or Placental Growth Factor (PGF);
comparing the levels VEGFB, THBS1 and/or PGF with the corresponding levels of VEGFB, THBS1 and/or PGF from a reference sample, said reference sample having a known clinical outcome; and
determining the prognosis for said patient based on said comparison.

The levels of Thrombospondin-1 (THBS1) and/or Placental Growth Factor (PGF) are preferably low plasma levels.

Also provided is a method of establishing a prognosis for a myocardially-infarcted patient, the method comprising:
assaying, post infarction, a body fluid sample from the patient for levels of Vascular Endothelial Growth Factor B (VEGFB) and levels of Thrombospondin-1 (THBS1) and/or Placental Growth Factor (PGF);
comparing the levels VEGFB, THBS1 and/or PGF with a control; and
determining the prognosis for said patient based on said comparison.

This may be achieved by comparing the levels VEGFB, THBS1 and/or PGF with the corresponding level of VEGFB, THBS1 and/or PGF in a reference sample;

wherein at least one of the following indicates an increased likelihood of an unfavorable prognosis for said patient:
lower levels of VEGFB in the assayed patient sample compared to the VEGFB control level;
higher levels of THBS1 in the assayed patient sample compared to the THBS1 control level; and/or
higher levels of PGF in the assayed patient sample compared to the PGF control level.

Also provided is a method of establishing a prognosis for a myocardially-infarcted patient, the method comprising:
assaying, post infarction, a body fluid sample from the patient for levels of Vascular Endothelial Growth Factor B (VEGFB) and levels of Thrombospondin-1 (THBS1) and/or Placental Growth Factor (PGF); and
questioning a previously built statistical program (also called “classifier”) with the levels of VEGFB, THBS1 and PGF;
   wherein
high level of VEGFB and low levels of THBS1 and PGF are associated with increased likelihood of developing said heart condition; and
the classifier will dictate whether the patient has an increased likelihood of developing said heart condition.

The levels of Thrombospondin-1 (THBS1) and/or Placental Growth Factor (PGF) are preferably low plasma levels.

The unfavorable prognosis is preferably that the patient has an increased likelihood of suffering a said heart condition.

The invention also provides a method of determining the likelihood of a myocardially-infarcted patient developing a heart condition, comprising the above steps.

According to another aspect, the present invention provides a method of identifying myocardially-infarcted patients having a reduced risk of developing a heart condition, comprising:
assaying, post infarction, a body fluid sample from the patient for levels of Vascular Endothelial Growth Factor B (VEGFB), Thrombospondin-1 (THBS1) and/or Placental Growth Factor (PGF);
comparing the levels VEGFB, THBS1 and/or PGF with the corresponding levels of VEGFB, THBS1 and/or PGF from a reference sample, said reference sample having a known clinical outcome; and
determining whether the patient has a reduced risk based on said comparison.

According to another aspect, the present invention provides a method of identifying myocardially-infarcted patients having a reduced risk of developing a heart condition, comprising:
assaying, post infarction, a body fluid sample from the patient for levels of Vascular Endothelial Growth Factor B (VEGFB), Thrombospondin-1 (THBS1) and/or Placental Growth Factor (PGF);
comparing the levels VEGFB, THBS1 and/or PGF with the corresponding levels of VEGFB, THBS1 and/or PGF from a control; and
determining whether the patient has a reduced risk based on said comparison.

This method may be amended to include comparing the levels VEGFB, THBS1 and/or PGF with the corresponding level of VEGFB, THBS1 and/or PGF in a reference;

wherein at least one of the following indicates a decreased likelihood of said patient suffering from said heart condition:
higher levels of VEGFB in the assayed patient sample compared to the VEGFB control level;
lower levels of THBS1 in the assayed patient sample compared to the THBS1 control level; and/or
lower levels of PGF in the assayed patient sample compared to the PGF control level.

This method may be amended to include:
questioning a previously built statistical program (also called “classifier”) with the levels of VEGFB, THBS1 and/or PGF;
   wherein
high level of VEGFB and low levels of THBS1 and PGF are associated with a decreased likelihood of developing said heart condition; and
the classifier will dictate whether the patient has a decreased likelihood of developing said heart condition.

The invention also provides a method of screening myocardially-infarcted patients for patients to assess the risk that each patient may have of developing a heart condition. This may be an increased or reduced risk.

The methods of the invention correlate the measurement of one or more biomarkers, with a better clinical outcome after MI. Most preferably, the biomarker is VEGFB and if the level thereof is high at day 1 post-MI, then this patient has a more favorable clinical outcome after MI. Most preferably, the biomarker is THBS1 and if the level thereof is low at day 1 post-MI, then this patient has a more favorable clinical outcome after MI. Most preferably, the biomarker is PGF and if the level thereof is low at day 1 post-MI, then this patient has a more favorable clinical outcome after MI.

It will be appreciated that the present methods are useful for establishing a prognosis in patients with MI, by correlating a combined assessment of multiple biomarkers, which, depending on their levels, can indicate a better clinical outcome after MI.

The invention may also be used in a personalized medicine setting. In a further aspect of the present invention, there is provided a method of providing or improving a patient's therapeutic strategy following MI, based upon identifying those patients at risk of developing a heart condition. This may be through the analysis of blood cell mRNA levels or plasma protein levels of VEGFB, THBS1 and/or PGF.

Diagnostic kits for use in the present invention are readily available for THBS1 and PGF, such as those available from R&D Systems. Inc. However, for VEGFB, it was necessary to construct our own diagnostic kit, as discussed below. Indeed, the only commercially available VEGFB kit (from USCNLIFE, VEGFB E0144h) was not sensitive enough to detect low VEGFB plasma levels. Using enhanced chemiluminescence as the detection method and an amplification step with biotin-streptavidin, the detection limit of our kit was 10 pg/mL whereas that of USCNLIFE kit, which uses a classical colorimetric detection, was found to be around 100 pg/mL.

Therefore, the invention also provides a method for assaying VEGFB levels in a sample, comprising:

(a) contacting the sample with at least one capture reagent immobilized to a support to form an immobilized capture reagent-sample complex;
(b) separating the sample from the at least one immobilized capture reagent;
(c) contacting the immobilized capture reagent-sample complex with a secondary antibody specific for VEGFB and optionally contacting the secondary antibody with a tertiary antibody specific for the secondary antibody;
(d) contacting the secondary or tertiary antibody with a binding molecule, such as streptavidin, conjugated to detection means; and
(e) measuring the level of the secondary or tertiary antibody bound to the capture reagents using the detection means.

Preferably, the capture reagent is an antibody, most preferably one that recognizes the same epitope as antibody mouse monoclonal clone 58013 against human VEGFB, said monoclonal antibody preferably binding specifically to VEGFB 167 and/or VEGFB 186.

Preferably, the secondary antibody is an antibody that recognizes the same epitope as antibody goat polyclonal that binds specifically to VEGFB 167 and/or VEGFB 186. Preferably, the tertiary antibody is a biotin-conjugated antibody specific for the secondary antibody, for instance a donkey anti-goat Ab. Preferably, the detection means comprises an alkaline phosphatase activity.

In a preferred embodiment, therefore, the invention provides a method for assaying VEGFB levels in a sample, comprising:

(a) contacting and, optionally incubating, the sample with a capture reagent immobilized to a solid support, wherein the capture reagent is an antibody that recognizes the same epitope as antibody mouse monoclonal clone 58013 against human VEGFB, said monoclonal antibody binding specifically to VEGFB 167 and VEGFB 186 to form an immobilized capture reagent-VEGFB complex;
(b) separating the sample from the immobilized capture reagents;
(c) contacting the immobilized capture reagent-VEGFB complex with a secondary antibody, wherein the secondary antibody is an antibody that recognizes same epitope as antibody goat polyclonal that binds specifically to VEGFB 167 and VEGFB 186;
(d) contacting the secondary antibody with a tertiary antibody, wherein the tertiary antibody is a biotin-conjugated donkey anti-goat antibody specific for the secondary antibody;
(e) contacting the tertiary antibody with streptavidin conjugated to alkaline phosphatase;
(f) measuring the level of VEGFB 186 or VEGFB 167 bound to the capture reagents using a detection means for the detectable antibody.

Additionally, we provide an ELISA kit to measure levels of VEGFB 186 and VEGFB 167 in biological fluids and its use as a diagnostic tool to identify the patients at risk of developing HF after MI.

The kit preferably comprises;

(a) at least one capture reagent immobilized to a support;
(b) a secondary antibody specific for VEGFB 186 and/or VEGFB 167;
(c) optionally, a tertiary antibody specific for the secondary antibody;
(d) a binding molecule, such as streptavidin, conjugated to detection means; and
(e) means for measuring the level of the secondary or tertiary antibody bound to the capture reagents using the detection means.

As above, preferably, the capture reagent is an antibody, most preferably one that recognizes the same epitope as antibody mouse monoclonal clone 58013 against human VEGFB, said monoclonal antibody preferably binding specifically to VEGFB 167 and/or VEGFB 186. Preferably, the secondary antibody is an antibody that recognizes the same epitope as antibody goat polyclonal that binds specifically to VEGFB 167 and/or VEGFB 186. Preferably, the tertiary antibody is a biotin-conjugated antibody specific for the secondary antibody, for instance a donkey anti-goat Ab. Preferably, the detection means comprises an alkaline phosphatase activity.

Said biological sample may, preferably, be isolated from a human subject and may be plasma or serum. It is also preferred that the immobilized capture reagents are coated on a microtiter plate. Preferably the detection is amplified by a chemiluminescent reagent. Purified human VEGFB 167 may be provided as an antigen standard.

We compared our VEGFB kit with the only commercially available VEGFB kit we found (USCNLIFE VEGFB E0144h). Our kit is more sensitive and therefore allows to measure VEGFB in more patients than the USCNLIFE kit.

According to a further aspect, the present invention provides a method of identifying myocardially-infarcted patients having a decreased risk of developing a heart condition, comprising:
assaying, post infarction, a body fluid sample from the patient for levels of Vascular Endothelial Growth Factor B (VEGFB), Thrombospondin-1 (THBS1) and/or Placental Growth Factor (PGF); and
questioning a previously built statistical program (also called “classifier”) with the levels of VEGBF, THBS1 and PGF;
   wherein
high level of VEGFB and low levels of THBS1 and PGF are associated with a decreased likelihood of developing said heart condition; and
the classifier will dictate whether the patient has a decreased likelihood of developing said heart condition.

We have a large database of acute MI patients with more than 20 clinical parameters and 4-months and 1-year follow-ups. Among other clinical parameters, the ejection fraction (EF) of the heart, which represents the capacity of the heart to pump blood into peripheral arteries, was measured by echocardiography the day of infarction, 4 months later and 1 year later. It is assumed that patients having an EF at 4-months≦40% suffer from remodeling whereas patients having an EF at 4-months>40% are recovering normally. Several protocols performed in our laboratory used the database to identify early biomarkers of the occurrence of HF after MI through different approaches. The biomarkers identified by two fundamentally different approaches were combined and the combination of the most predictive biomarkers was defined as the “prognostic set”.

The first approach involved DNA microarray technology. Biosignatures or gene expression profiles of circulating blood cells were analyzed from blood samples withdrawn the day of MI. This technology allowed the identification of differentially regulated genes between two groups of patients with extreme phenotypes, i.e. one group of patients having a favorable clinical outcome after MI (high EF group, EF>40%) and one group having an unfavorable outcome after MI (low EF group, EF≦40%). We have characterized the biosignatures of blood cells from 32 patients, 16 from each high EF and low EF group. Using the SAM algorithm (Statistical Analysis of Microarrays) and a fold-change of 1.3, 525 genes were found differentially expressed between the 2 groups of patients (False Discovery Rate of 24.5%). Among these genes, 9 had a significant prognostic value for HF.

The second approach was based on a bioinformatic characterization of a protein-protein interaction network of angiogenesis in human MI. Indeed, angiogenesis is one of the beneficial healing processes that take place in the heart after MI and a defect in angiogenesis can lead to HF. The network was built with annotated protein-protein interactions from the Human Protein Reference Database. This global network consisted of 556 nodes (i.e. proteins) and 686 edges (i.e. interactions). After subsequent network-based and gene expression analyses, 38 network-derived genes showed a significant prognostic value. Interestingly, the combination of the gene expression-based classification models with the network-based classification models yield to a reduced number of candidate biomarkers with a greatly improved prognostic value than each approach considered separately. The area under the curve (AUC), which represents the prognostic capacity of the biomarkers, was between 0.56 and 0.72 for the gene expression-based classification model, and between 0.56 and 0.73 for the network-based classification model. When the two models were combined, a set of 3 biomarkers with and AUC of 0.82 (i.e. with a strong prognostic value for the occurrence of HF) was implemented: this set is called here the “prognostic set”. These 3 biomarkers were: thrombospondin-1 (THBS1), placental growth factor (PGF or PlGF), and Vascular Endothelial Growth Factor B (VEGFB). While THBS1 has anti-angiogenic properties, VEGFB and PGF owe their pro-angiogenic capacities to stimulation of the growth and multiplication of vascular endothelial cells.

Then, we verified that the differences in gene expression observed at the mRNA level by microarrays were effective at the protein level. For this purpose, plasma levels of the 3 biomarkers were measured by enzyme-linked immunosorbent assay (ELISA). These experiments attested that protein levels of THBS1 and PGF were significantly distinguishable between patients with high and low EF.

Since VEGFB ELISA kits commercially available were not found sensitive enough to detect VEGFB in our plasma samples, we designed our own kit which allows the quantification of VEGFB in biological fluids such as human plasma.

By “favorable outcome,” it will be understood that this means a lower risk of the patient going on to develop a heart condition, such as Heart Failure and/or suffer from Left Ventricular remodeling. It is believed that Right Ventricular Remodelling is less relevant, so this is not preferred.

By “unfavorable outcome,” it will be understood that this means a higher risk of the patient going on to develop a heart condition, such as Heart Failure and/or suffer from Left Ventricular remodeling.

It will also be understood that whilst the risks of Heart Failure and Left Ventricular Remodeling are associated, these are separate conditions and, therefore, a patient could suffer one, but not the other. Therefore, increased risk of either Heart Failure or Ventricular Remodeling is unfavorable.

It will be appreciated that the RNA sequences given in the sequence listing comprise Thymine (T) as this is how they are represented on the NCBI website. In each case, it is clear that replacement of T with Uracil (U) is contemplated.

### BRIEF DESCRIPTION OF THE DRAWINGS

FIG. 1 is an illustration of the microarrays data interpreted with SAM algorithm.

FIG. 2 shows the protein-protein interaction network of angiogenesis in human MI.

FIG. 3 illustrates the strategy used for the combined analysis of the gene expression-based classification models with the network-based classification models.

FIG. 4 shows a heat-map illustrating the differences in the expression (microarrays) of the biomarkers among patients with high (H) and low (L) ejection fraction.

FIG. 5 shows quantile-quantile plots illustrating the relationship between the ejection fraction and the expression of the biomarkers assessed by microarrays and ELISA.

FIG. 6 shows scatter-plots illustrating the relationship between the ejection fraction and the expression of the biomarkers assessed by microarrays and ELISA.

FIG. 7 represents the evolution of VEGFB plasma levels between the day of infarction (day 0) and the day after (day 1). Whereas plasma VEGFB decreases between day 0 and day 1 in patients with low EF (−10%), patients with high EF have increasing VEGFB levels (+15.4%).

FIG. 8A Shows expression values of VEGFB using quantitative PCR and microarrays for high EF and low EF patients

FIG. 8B shows the significant correlation observed between VEGFB expression and ejection fraction.

FIG. 9 shows VEGFB levels between high and low EF groups at day 0, day 1 and day 2 after MI.

Table 1 is a summary of the predictive performances of classification models based on mRNA levels of VEGFB, THBS1 and PGF.

Table 2 is a summary of the conclusions reached in the experiments underling the present invention.

Table 3 is a summary of the statistics performed to compare the levels of the VEGFB, THBS1 and PGF between patients with high EF and patients with low EF.

Table 4 is a summary of the statistics of the comparison between mRNA levels of the 3 biomarkers in the two groups.

Table 5 is a summary of the predictive performance of NT-pro-BNP.

Table 6 is a list of 28 angiogenic genes differentially expressed between high and low EF groups.

Table 7 shows prediction performances using two machine learning models.

### DETAILED DESCRIPTION

Heart Failure (HF) is the major complication of myocardial infarction (MI). Recent data showed that 63% of the patients develop HF in the 6 years following MI. Angiogenesis is a key phenomenon involved in the repair of the myocardium after MI. Angiogenesis is tightly regulated by a balance being governed by a large number of angiogenic factors, some being pro- and others being anti-angiogenic. A deregulation of this balance can lead to inappropriate angiogenesis and can set the stage for the development of HF after a MI episode.

Recent reports clearly indicate that the early identification of patients prone to suffer from HF after MI may significantly improve the tailoring of the therapeutic strategies to the individual patient (“personalized medicine”). Like many other cardiovascular disorders, HF is a multifactorial disease. Thus the use of single biomarkers to predict the occurrence of HF after MI has limited value. The multiplication of prognostic biomarkers may therefore be of interest to finely predict HF.

First, we hypothesized that a set of prognostic biomarkers could be identified through the analysis of the biosignatures of circulating blood cells. Second, we hypothesized that a protein-protein interaction network-based approach may also have the potential to highlight prognostic biomarkers of HF. And third, we tested whether the combination of the data obtained by these two independent approaches would allow achieving a higher level of prognosis than each approach taken separately.

For this purpose, we selected two groups of patients (n=16 per group) with MI, one group of patients having a favorable outcome after MI (Ejection Fraction (EF)>40%) and one group of patients having an unfavorable outcome (EF≦40%). This strategy to select “extreme phenotypes” was chosen to increase the chances of finding differentially expressed genes between the two groups without the need of a large sample size. RNA was extracted from whole cells isolated from the peripheral blood of these patients. Biosignatures were determined by microarray profiling. After several normalization, filtering and statistical procedures, a set of 525 genes were found to be differentially expressed between the two groups of patients (fold-change 1.3; false discovery rate 24.5%). Among those, a cluster of 47 genes with moderate prognostic value was identified by classification models with a maximum AUC of 0.72. Further filtering of these genes led to a cluster of 9 genes with equivalent prognostic value (AUC 0.68).

In an attempt to increase the strength of the prediction afforded by genes retrieved from microarray experiments, and considering the importance of angiogenesis in the repair of the heart after MI, a protein-protein interaction network of angiogenesis in human MI was drawn. This network was assembled by extracting genes known to be involved in this process and corresponding (curated) protein-protein interactions from public databases. Clustering analysis of this network reported a module significantly associated with cell growth and growth regulation. Within this cluster, 38 genes were found to be significantly differentially expressed between the EF classes. Different, independent classifiers built with these 38 genes reported a moderate prognostic value (max. AUC=0.73), equivalent to that obtained from microarrays. Interestingly, further filtering of these genes (correlation-based feature selection) yielded a set of 3 genes with a stronger prognostic value (AUC=0.82 using an instance-based learner, Table 1). These 3 genes, VEGFB, THBS1 and PGF, are viewed here as a new “prognostic set” of biomarkers of HF. Differential expression of these 3 genes was validated by quantitative PCR. In addition, plasma levels of the 3 biomarkers were measured.

In one finding, we showed that patients able to mount a significant response to MI, characterized by high mRNA or plasma protein levels of Vascular Endothelial Growth Factor B (VEGFB) and low plasma levels of Thrombospondin-1 (THBS1) and Placental Growth Factor (PGF or PlGF), have a low susceptibility to develop HF and/or undergo Ventricular Remodeling. Measurement of the plasma levels of these three biomarkers can therefore serve as a useful tool for predicting the occurrence of HF and/or Ventricular Remodeling after MI.

TABLE 1
Predictive performance of classification models based on
mRNA levels of the 3 biomarkers.

| | Classification | Typical | |
|---|---|---|---|
| Input type | model | accuracy* (%) | AUC* |
| SAM-based biomarkers only | K* | 65 | 0.63 |
| Network-based biomarkers | K* | 84 | 0.82 |
| SAM-based biomarkers only | SVM | 68 | 0.68 |
| Network-based biomarkers | SVM | 75 | 0.75 |

*Based on leave-one-out cross-validation.
Potential biomarkers used: SAM-derived (9 genes) and network-based (3 genes).
AUC: Area under the (ROC) curve;
SVM: Support Vector Machine.

Therefore, we propose a new strategy to identify the patients at risk of developing HF after MI, based on the measurement of a cluster of 3 biomarkers, VEGFB, THBS1 and PGF. These measurements can be made from RNA extracted from blood cells or from plasma levels of the corresponding proteins.

Table 2 summarizes the particular findings behind the present invention.

TABLE 2
Association between the mRNA and plasma levels of the
3 biomarkers and the EF.

| | |
|---|---|
| High EF | Low EF |
| (favorable | (unfavorable |
| outcome) | outcome) |

| | mRNA | Plasma | mRNA | Plasma |
|---|---|---|---|---|
| VEGFB | High | High | Low | Low |
| THBS1 | Low | Low | High | High |
| PGF | Low | Low | High | High |

mRNA levels are measured by microarrays in blood samples harvested the day of MI Plasma levels are measured by ELISA one day after MI.

All references cited herein are hereby incorporated in their entirety to the extent that they do not conflict with the present invention.

The invention will now be described in further detail in the accompanying non-limiting examples.

### EXAMPLES

### Example 1

### Patients and Methods

### Patients

Patients with acute MI were treated with primary percutaneous coronary intervention. Acute MI was defined by the presence of chest pain <12 hours with significant ST segment elevation and positive cardiac enzymes. Blood samples were obtained at the time of mechanical reperfusion (for microarrays and quantitative PCR analyses) and the day after MI (for plasma levels determination). All patients signed an informed consent.

### Microarrays

To increase our chances to detect relevant biomarkers in the context of ventricular remodeling, we selected two groups of patients having “extreme” phenotypes after MI, namely patients that evolved favorably after infarction (EF≧45%, average 61%) and patients that evolved unfavorably (EF≦40%, average 33%). Each group contained 16 patients.

Total RNA was extracted from whole blood cells by the PAXgene™ technology. Blood withdrawn in PAXgene™ blood RNA tubes (PreAnalytix®, BD Europe, Erembodegem, Belgium) was stored at −20° C. until RNA extraction. Extraction was performed with the PAXgene™ Blood RNA kit (Qiagen, Courtaboeuf, France) according to the manufacturer's instructions. RNA quantity was measured using the ND-1000 spectrophotometer (NanoDrop® Technologies, Wilmington, USA). RNA quality was assessed using the 2100 Bioanalyzer® apparatus (Agilent Technologies, Massy, France) with the RNA 6000 Nano chips. Only high quality RNA (OD₂₆₀/OD₂₈₀>1.9 and OD₂₆₀/OD₂₃₀>1.7) and undegraded RNA was considered for further analysis.

A common reference RNA (Universal Human Reference RNA, Stratagene Europe, Amsterdam, The Netherlands), a mixture of RNA from 11 cell lines was used in conjunction with patient's RNA in all following steps in order to provide an internal reference standard for comparisons of relative gene expression levels across arrays.

Messenger RNAs were amplified using the Amino Allyl MessageAmp™ kit (Ambion®, Cambridgeshire, United Kingdom) according to the manufacturer's protocol, starting with one μg of total RNA. Five μg of each amino allyl aRNA were labeled with Cy3 or Cy5 (Amersham, Buckinghamshire, United Kingdom). Dye coupling to amino allyl aRNA was measured using the ND-1000 NanoDrop® spectrophotometer. Dye coupling yield >5% was a prerequisite for further analysis. 750 ng of each amino allyl aRNA labeled Cy3 or Cy5 (reference RNA or donor RNA) were combined and hybridized on pangenomic oligonucleotide microarrays containing 25,000 genes (Genomic Platform, Illkirch, France). Four microarrays per patient were hybridized and a dye-swap was performed (2 microarrays patient-Cy3/reference-Cy5 and 2 microarrays patient-Cy5/reference-Cy3). Hybridization steps were performed using the Agilent Technologies system. Briefly, RNA was fragmented with a fragmentation buffer before mixing with a hybridization buffer. Microarrays were blocked with 50 mM ethanolamine in 50 mM borate buffer pH=9.0. Agilent's hybridization chambers and rotating oven were used for hybridization at 60° C. for 17 h at 4 rpm. Microarrays were washed for 10 min in 6×SSC, 0.005% Triton X-102, for 5 min in 0.1×SSC, 0.005% Triton X-102, and were then dried by centrifugation before scanning using an Axon 4000B microarray scanner and the GenePix Pro 6® software (Molecular Devices, Berks, UK). Self photomultiplicator gain adjustment and 0.1% saturated spots were allowed during scanning.

Spot finding and raw-data quantification of all four microarrays for each patient were performed in a batch analysis using the MAIA® freeware (Institut Curie, France). This software assigns each spot with nine quality parameters that allow for determination of “good quality spots” among the four microarrays. Only good spots are kept for further analysis. A Lowess non-linear normalization step was performed with the Acuity® software (Molecular Devices) to compensate for uneven Cy3-Cy5 distribution. Normalized log ratio of Cy3/Cy5 was used in subsequent steps. A filtering step was then performed to remove genes that were not present in at least three microarrays out of four. The quality and reproducibility of each of the four microarrays per patient were evaluated using ANOVA, correlation coefficients and Self Organizing Maps drawn with the Acuity® software. Data are stored in the Web-based Microarray Data manager MEDIANTE.

Before statistical analysis, genes not present in at least 50% of the patients were filtered out. Supervised analysis was performed using two complementary approaches. The first approach involved the Significance Analysis of Microarrays (SAM) software which correlates gene expression with an external variable such as EF value. Two-class unpaired test was used. Gene missing-values imputation was performed via a K-Nearest Neighbour algorithm using 10 neighbours.

### Protein-Protein Interaction Network

A set of core genes, known to be associated with angiogenesis in myocardial infarction, was retrieved from the Entrez-Gene database; query: “human AND heart AND angiogenesis AND myocardial AND infarction”. Annotated protein-protein interactions associated with these core genes were retrieved from the Human Protein Reference Database (HPRD).

A network clustering analysis was implemented to identify potential functional network modules. Clusters were identified by the (Cytoscape plug-in) MCODE network clustering algorithm.

### Biochemical Assays for THBS1, PGF and NT-pro-BNP

Plasma levels of THBS1 and PGF were measured in samples from 46 patients by ELISA using the Quantikine DTSP10 and DPG00 kits, respectively (R&D Systems, Oxon, UK). Detection limits of the assays were 0.35 ng/mL for THBS1 and 7 p/mL for PGF. Plasma level of pro-BNP (N-Terminal-pro-BNP, NT-pro-BNP) was measured using the Elecsys 2010 immunological device (Roche Diagnostics, Meylan, France). Detection limit of the assays was 20 pg/mL.

### Set up of the VEGFB Diagnostic Kit

A sandwich ELISA was developed to detect VEGFB 167 and VEGF-B 186. Microtiter plates (Lumitrac 600, Greiner, Belgium) are coated with 100 μl of mouse anti-VEGF-B monoclonal antibody (2 μg/ml in PBS, MAB751, R&D systems, UK) overnight at 4° C. After three washings, plates are blocked for 1 hour with 300 μl of 5% BSA-PBS at 500 rpm and room temperature. A standard curve is produced from 2000 pg/ml to 15.6 pg/mL with human VEGFB 167 (751-VE, R&D Systems) in 1% BSA-PBS. After blocking, plates are washed three times and incubated for 2 hours with 100 μl of plasma, blank or standards at 500 rpm and room temperature. After three washes, 100 μl of goat polyclonal VEGF-B antibody (400 ng/ml in 1% BSA-PBS, AF751, R&D Systems) are added to each well and plates are incubated for 1 hour at 500 rpm and room temperature. After three washes, 100 μl of biotin conjugate donkey anti-goat antibody (1:27500 in 1% BSA-PBS, 705-065-147, Jackson, USA) are added to each well and plates are incubated for 1 hour at 500 rpm and room temperature. After three washes, 100 μl of streptavidin conjuged to alkaline phosphatase (2 μg/ml in 1% BSA-PBS, 016-050-084, Jackson) are added to each well and plates are incubated for 1 hour at 500 rpm and room temperature. Plates are washed four times with Tris-Buffered Saline added with Tween 20 (pH 7.5) before incubation with 100 μl per well of Lumiphos 530™ (Lumigen, USA) for 30 minutes at 500 rpm, room temperature and protected from light. Chemiluminescence is detected using a Polarstar Optima (BMG Labtech, Paris, France).

### Statistical Analysis

For each set of inputs, different standard statistical and machine learning classifiers were evaluated, e.g. Prediction Analysis for Microarrays (PAM), Support Vector Machine (SVM) and the K* techniques. The K* is an instance-based model that classifies a new sample based on the class information provided by its most relevant (or nearest) neighbors in a training dataset. K* applies an entropy-based distance measure to estimate the neighborhood set. Models were implemented with global blend=20, and average column entropy curves for estimating missing values.

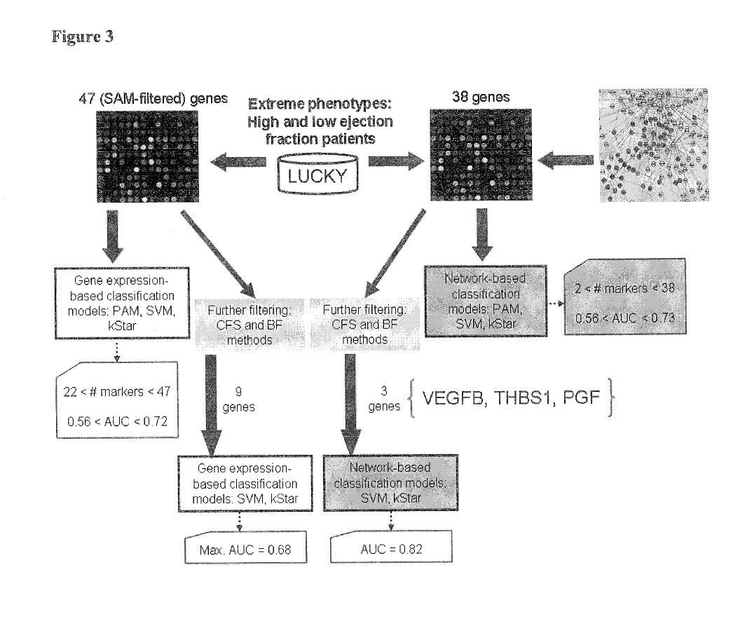

Further filtering on the network-based gene data was implemented with the correlation-based feature selection (CFS) algorithm using the “best first search” (BF) strategy (FIG. 3). The CFS is a filter feature selection method that finds subsets of features (i.e. genes) that maximizes gene-class correlation while minimizing gene-gene correlation. Filter feature selection methods are implemented independently of any classification model. The BF strategy was based on a greedy hill-climbing augmented with a subset backtracking.

Classification evaluation results were estimated using the leave-one-out cross-validation (LOO) strategy, as well as 10-fold cross-validation. The estimated areas under the curves (AUC) of the cross-validated ROC (receiver operating characteristic curve) were used to summarize the estimated classification performance of the classifiers. Statistical differences between EF groups (on the basis of each of the biomarkers) was implemented through Student's t test, and corroborated with non-parametric tests. Correlations between these biomarkers and the EF values were estimated with standard Pearson coefficients (Table 2).

### Software

Machine learning models implementation and statistical evaluation were performed with the Weka (v. 3.4) data mining platform. Hierarchical clustering was implemented using unweighted pair-group method with arithmetic averages and correlation coefficients. Clustering visualization was performed with GEPAS. Statistical significance tests, Pearson correlation values, and graphical plots were generated with the Statistica package (v. 6.0).

### Results

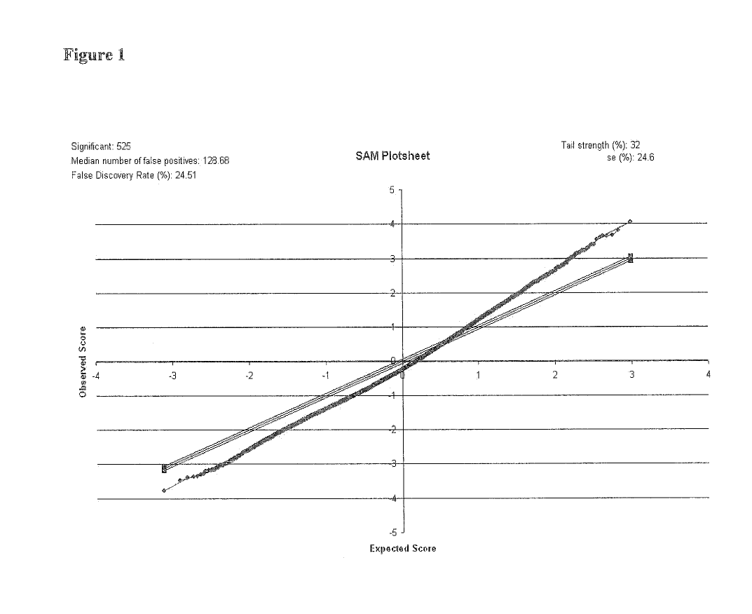

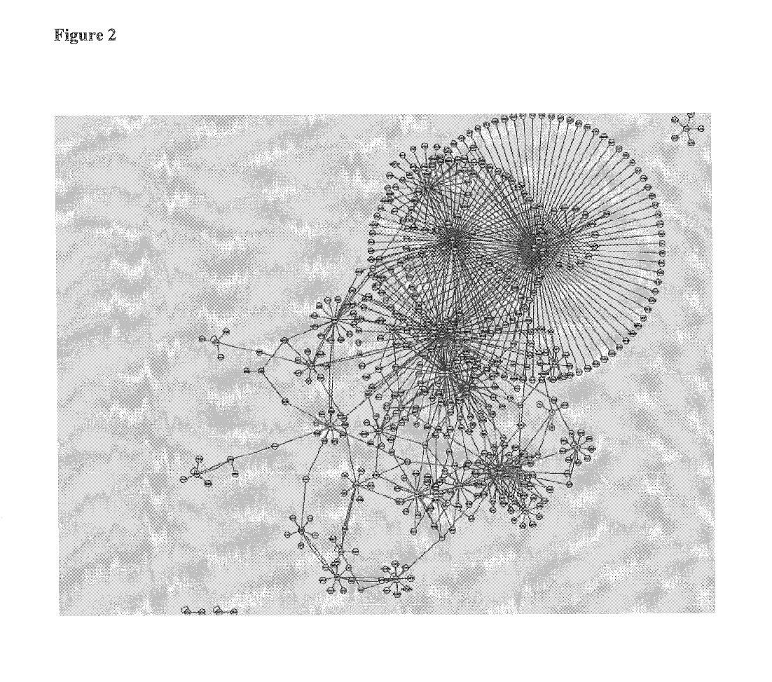

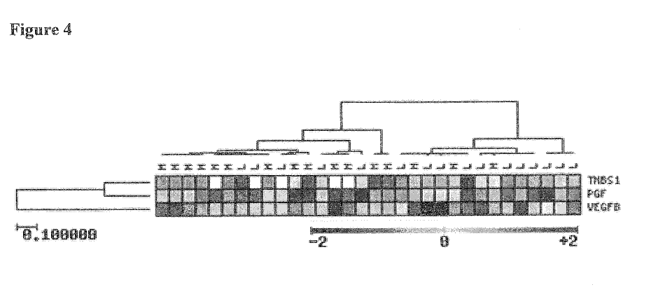

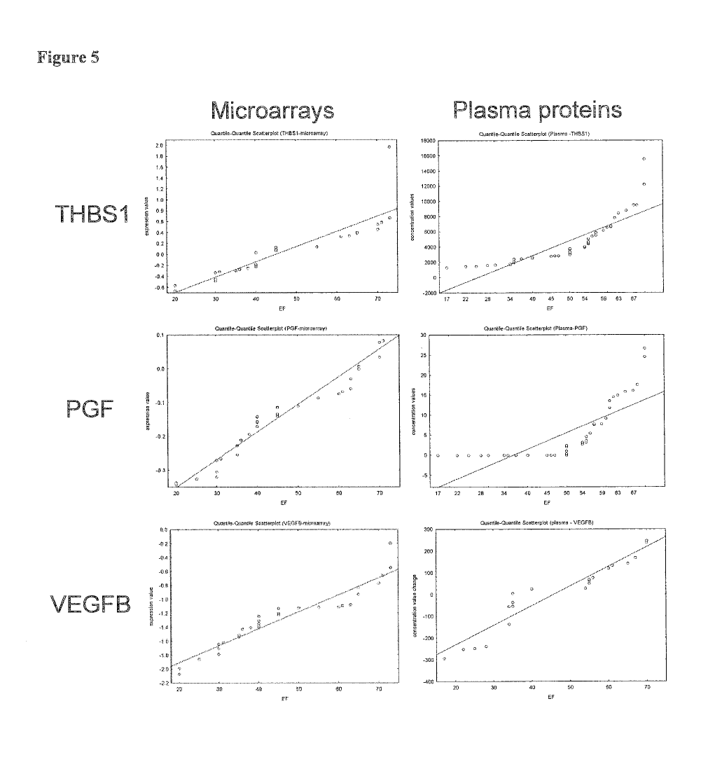

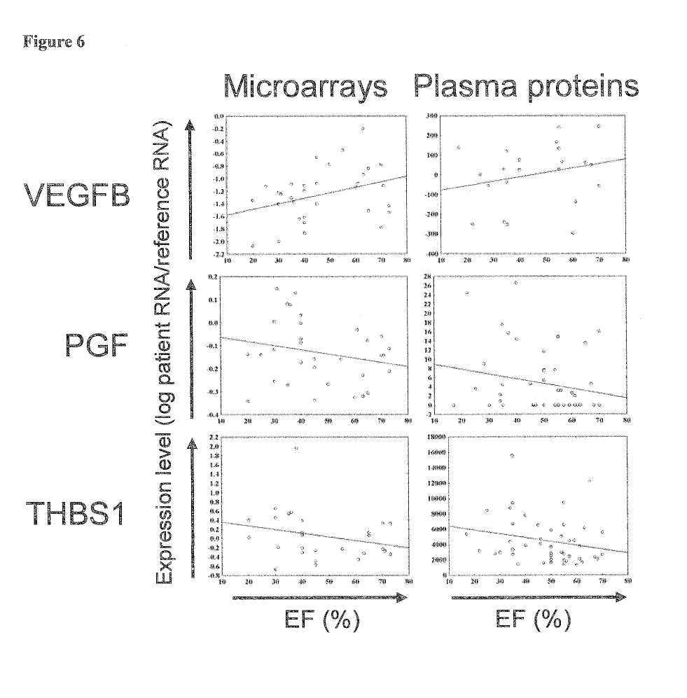

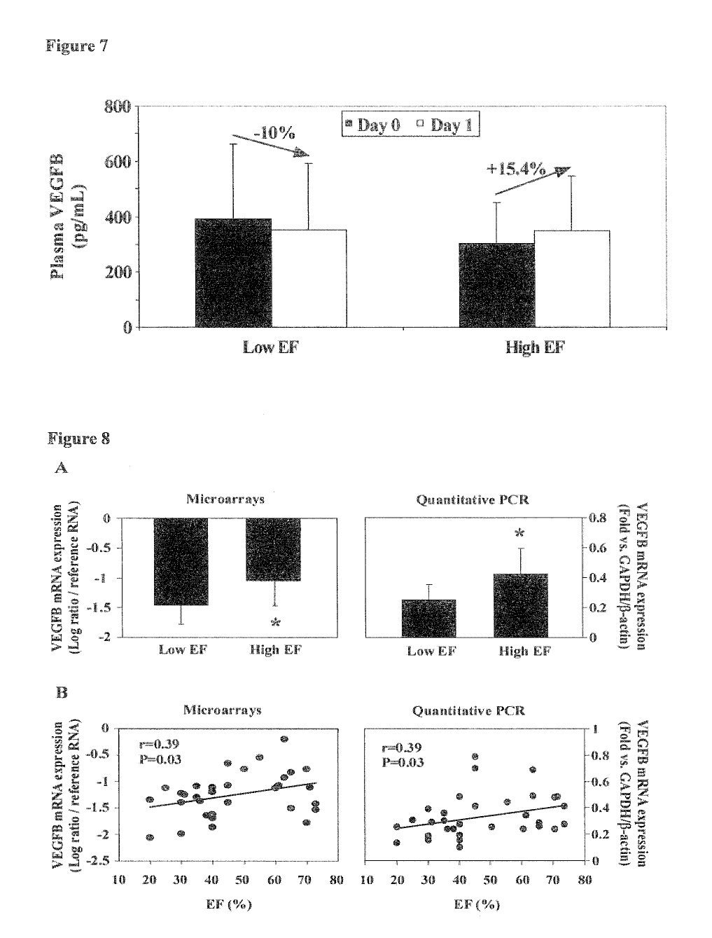

1. Statistical Analysis of Microarrays (SAM) plot showing that 525 genes are differentially expressed between low and high EF groups (FIG. 1). A threshold for fold-change of 1.3-fold was selected and a FDR of 24.5% was obtained. Red dots represent genes up-regulated in the low EF group, green genes up-regulated in the high EF group, and black dots represent genes whose fold-change is <1.3 between the two groups.
2. Protein-protein interaction network of angiogenesis in human MI (FIG. 2). The resulting network consisted of 556 nodes (proteins) and 686 edges (interactions). Network clustering analysis consistently highlighted the existence of a network cluster (53 proteins) with a highly significant over-representation of (Gene Ontology) biological processes relating to cell growth and growth regulation.
3. FIG. 3 shows that combined analysis of the gene expression-based classification models with the network-based classification models allowed the identification of 3 genes with higher prognostic value (max. AUC=0.82) than gene expression-based classification models alone (0.56<AUC<0.72) or network-based classification models alone (0.56<AUC<0.73). The highest prognostic performances (based on the 3 genes) obtained to date have been obtained with the instance-based learning model K*.
4. A heat-map illustrates the differences in the expression (obtained by microarrays) of the biomarkers among patients with high (H) and low (L) ejection fraction (FIG. 4). Colors (red, pink, light blue, dark blue) show the range of expression values (high, moderate, low, lowest). White color indicates undetectable values. VEGFB is clearly more expressed in the group of patients having a high EF whereas THBS1 and PGF are more expressed in the low EF group.
5. Quantile-quantile plots (FIG. 5) and scatter-plots (FIG. 6) illustrating statistical dependencies between the ejection fraction and the expression of the biomarkers assessed by microarrays and ELISA. The linear relation shown suggests that these variables follow similar data distributions. Results from Student's t-test and linear coefficient correlation corresponding to these plots are summarized in Table 3. VEGFB is positively correlated with the EF, whereas THBS1 and PGF are negatively correlated with the EF. Concordant results are obtained with microarrays and ELISA for THBS1 and PGF. VEGFB mRNA level was found significantly higher in low EF patients than in low EF patients by the microarray technique.

TABLE 3

| Gene | Microarrays | Plasma proteins |
|---|---|---|
| THBS1 | t = 2.4, p = 0.02 | t = 2.1, p = 0.04 |
| | r = −0.3, p = 0.2 | r = −0.2, p = 0.1 |
| PGF | t = 2.8, p = 0.01 | t = 2.2, p = 0.04 |
| | r = −0.2, p = 0.2 | r = −0.2, p = 0.2 |
| VEGFB | t = 3.1, p = 0.004 | t = −1.3, p = 0.20 |
| | r = 0.3, p = 0.05 | r = 0.2, p = 0.3 |

t: t statistic
r: linear correlation coefficient

Statistics performed on mRNA levels measured by microarrays in blood samples harvested the day of MI and plasma levels measured by ELISA one day after MI.
6. Descriptive statistics of the mRNA levels of the 3 biomarkers in the two groups of HF patients showing the higher level of VEGFB mRNA and the lower levels of THBS1 and PGF mRNA in high EF patients compared to low EF patients (Table 4). Also provided in this table are theoretical thresholds for mRNA levels associated with either a high EF (>40%) or a low EF (≧40%) as determined from FIGS. 5 and 6. For instance, in the population of HF patients used in this study, a patient having a level of VEGFB mRNA higher than −1.4, a level of THBS1 mRNA lower than 0 and a level of PGF mRNA lower than −0.1 was more prone to have a high EF. Conversely, a patient having a level of VEGFB mRNA lower than −1.4, a level of THBS1 mRNA higher than 0 and a level of PGF mRNA higher than −0.1 was more prone to have a low EF. In other words the levels of biomarkers were compared to a reference sample after the reference sample had been calibrated against a range of clinical outcomes. It is important to mention that the combination of the 3 biomarkers rather than each biomarker alone or a combination of 2 biomarkers is more accurately associated with the EF.

TABLE 4
Descriptive statistics of the mRNA levels of the 3 biomarkers
in the two groups.

| | High EF | | Low EF | | |
|---|---|---|---|---|---|
| | (favorable | | (unfavorable | | |
| | outcome) | | outcome) | | |
| | mean ± SD | Thresh- | mean ± SD | Thresh- | |
| | (min/max) | old | (min/max) | old | T stat |
| VEGFB | −1.05 ± 0.42 | >−1.4 | −1.46 ± 0.32 | <−1.4 | t = 3.1 |
| | (−1.78/−0.20) | | (−2.07/−1.09) | | p = 0.004 |
| THBS1 | −0.13 ± 0.28 | <0 | 0.29 ± 0.58 | >0 | t = 2.4 |
| | (−0.57/0.33) | | (−0.66/1.97) | | p = 0.02 |
| PGF | −0.19 ± 0.10 | <−0.1 | −0.07 ± 0.15 | >−0.1 | t = 2.8 |
| | (−0.34/−0.03) | | (−0.34/0.15) | | p = 0.01 |

t: t statistic
p: probability value
SD: standard deviation

7. Additional testing of the classifier using plasma levels of VEGBF, THBS1 and PGF measured 1 day after MI in the same 32 patients that have been used to build the classifier yielded an AUC of 0.75. This suggests that the prediction performance of the classifier is good even when using a different dataset encoding other types of measurements (e.g. plasma levels).
8. Since the only established biomarker of HF is pro-BNP, the prognostic performance of the 3 biomarkers set disclosed here was compared with the prognostic performance of NT-pro-BNP. The prognostic performance of the plasma level of NT-pro-BNP, measured 1 day after MI, was moderate (AUC=0.63, Table 5). Therefore the set of 3 biomarkers disclosed here clearly outperformed the prognostic value of NT-pro-BNP.

TABLE 5
Predictive performance of NT-pro-BNP.

| Input type | Classification model | Typical accuracy* (%) | AUC* |
|---|---|---|---|
| NT-pro-BNP | K* | 50 | 0.52 |
| NT-pro-BNP | SVM | 63 | 0.63 |

*Based on leave-one-out cross-validation.
AUC: Area under the (ROC) curve;
SVM: Support Vector Machine.

### Example 2

### Patients and Methods

Patients with acute MI were enrolled in a national MI registry and treated with primary percutaneous coronary intervention. Acute MI was defined by the presence of chest pain<12 hours with significant ST elevation and increase in creatine kinase and troponin I to greater than 2 fold upper limit of normal. Blood samples were obtained at the time of mechanical reperfusion (for RNA and plasma isolation), one day or two days after MI (for plasma). The protocol has been approved by the local ethics committee and informed consent has been obtained from all subjects.

The validation cohort of 290 MI patients was from a prospective study conducted at the University Hospitals of Leicester NHS Trust (UK). Echocardiography was carried out at discharge and 6 months after MI. LV end diastolic volume (LVEDV) was estimated using the bi-planar modified Simpson's rule from apical two and four chamber views. The degree of LV remodelling was assessed from the change in LVEDV (□EDV) between discharge and follow-up.

### Microarrays

Transcriptomic profiles of whole blood cells were obtained using oligonucleotide microarrays representing 25,000 genes. Data are available at the Gene Expression Omnibus database (www.ncbi.nlm.nih.gov/geo/) under the accession number GSE8723. Supervised analysis was performed using the Significance Analysis of Microarrays (SAM) software. Statistical significance of the over representation of Gene Ontology (GO) terms in gene sets was estimated with the DAVID database. Heat maps were drawn using the Gene Set Enrichment Analysis (GSEA) software.

### Measure of VEGFB Expression

VEGFB mRNA expression in blood cells obtained the day of MI was determined by quantitative PCR. A homemade sandwich ELISA was developed to measure plasma levels of VEGFB.

### Patient Classification Models

Support Vector Machine (SVM) and the K* computational classification models were evaluated to test the prognostic significance of VEGFB expression levels. The SMO (sequential minimal optimization) algorithm for training SVM classifiers was implemented with the following parameters: complexity parameter C=1.0, epsilon=1.0E-12, exponent of polynomial kernel=1.0. Models were implemented with global blend=20, and average column entropy curves for estimating missing values. Classification evaluation results were estimated using the leave one out cross validation (LOO) strategy. The area under the receiver operating characteristic curve (AUC) was used to summarize the estimated classification performance of the classifiers.

### Statistical Analysis

Comparisons between the means of two groups of patients were performed with two-tailed unpaired t-test for Gaussian data and Mann-Whitney test for non Gaussian data. Categorical variables were compared using the Fisher exact test. Correlation between biomarker levels and the EF class was estimated with the Spearman test. Machine learning models implementation was performed with the Weka (v. 3.4) data mining platform. Hierarchical clustering was implemented using unweighted pair-group method with arithmetic averages and correlation coefficients. Clustering visualization was performed with GEPAS. Statistical significance tests were generated with the Statistica package (v. 6.0). A P value<0.05 was considered statistically significant.

### Results

### Patient Selection and Characteristics

We enrolled patients presenting with acute ST elevation MI, treated by mechanical reperfusion. For transcriptomic analyses, two groups of 16 patients with acute MI were selected based on their EF 1 month after MI. One group of patients had a preserved LV systolic function with high EF after MI (>40%, median 63%, range 45-73), and the other group impaired LV function with low EF (≦40%, median 35%, range 20-40).

### Transcriptomic Analysis of Blood Cells

Gene expression profiles of whole blood cells isolated at the time of reperfusion were obtained using 25,000 genes microarrays. Among these, 525 genes were found differentially expressed by SAM between high EF and low EF patients with a 1.3 fold change threshold and a false discovery rate of 24.5%. 226 genes were up regulated in the high EF group and 299 were up-regulated in the low EF group. Out of the 525 genes, GSEA retrieved the 50 genes most significantly associated with one or the other group of patients.

Angiogenic Genes Associated with Clinical Outcome after MI

Following our working hypothesis that angiogenesis may play a significant role in cardiac repair after MI, we aimed to identify from the 525 genes differentially expressed between high and low EF patients those genes related to angiogenesis. For this purpose, we retrieved from the Entrez Gene database a list of 494 genes known to be related to angiogenesis in humans with the following query: “angiogenesis” AND “homo sapiens”. Of the 525 differentially expressed genes, 28 were found in this set of 494 angiogenic genes: 20 up regulated in the low EF group and 8 up regulated in the high EF group (Table 6).

TABLE 6
List of 28 angiogenic genes differentially expressed between
high and low EF groups.
Over-expressed in low EF group

| Pro | | | Anti | | |
|---|---|---|---|---|---|
| angiogenic | Fold- | | angiogenic | Fold- | |
| (n = 15) | change | q-value (%) | (n = 6) | change | q-value (%) |
| BMX | 1.90 | 11.36 | CLU | 1.50 | 4.97 |
| PBEF1 | 1.73 | 20.21 | THBS1 | 1.44 | 5.88 |
| FOS | 1.66 | 4.09 | ITGB1 | 1.34 | 25.29 |
| PFKFB3 | 1.65 | 0.00 | MAPK14 | 1.31 | 24.72 |
| CD55 | 1.65 | 5.53 | STAT1 | 1.31 | 25.56 |
| HIF1A | 1.63 | 0.00 | MME | 1.31 | 20.21 |
| IL8 | 1.59 | 7.51 | | | |
| PTGS2 | 1.55 | 11.36 | | | |
| TGFBR1 | 1.50 | 9.87 | | | |
| THBS1 | 1.44 | 5.88 | | | |
| SLC2A3 | 1.40 | 0.00 | | | |
| ERO1L | 1.32 | 28.34 | | | |
| PLAUR | 1.31 | 5.88 | | | |
| ADM | 1.31 | 25.06 | | | |
| B2M | 1.30 | 28.12 | | | |

Under-expressed in low EF group

| Pro | | | Anti | | |
|---|---|---|---|---|---|
| angiogenic | Fold- | | angiogenic | Fold- | |
| (n = 4) | change | q-value (%) | (n = 4) | change | q-value (%) |
| VEGFB | 0.74 | 4.97 | SOD1 | 0.70 | 11.36 |
| RHOC | 0.74 | 8.70 | MAGED1 | 0.75 | 9.31 |
| CX3CR1 | 0.76 | 17.27 | ANXA2 | 0.76 | 17.27 |
| ATP5B | 0.80 | 21.32 | BAI1 | 0.76 | 17.27 |

q value: the lowest false discovery rate at which the gene is called significant (like ‘p value’ adapted to the analysis of a large number of genes).
Note:
the total number of genes in this table is 28 since thrombospondin 1 (THBS1) is both pro and anti angiogenic.

A heat map drawn with these 28 genes showed that clinical outcome after MI is associated with a distinct biosignature linked to angiogenesis.

In an attempt to evaluate whether this biosignature was associated with stimulation or repression of angiogenesis, we questioned the Entrez Gene database for pro or anti angiogenic properties of the 28 angiogenic genes found differentially expressed between the 2 groups of MI patients. As shown in Table 6, the balance between known pro and anti angiogenic factors tends to lean toward the pro angiogenic side for low EF patients, although it can only be speculated that this is associated with stimulation of angiogenesis.

We then further narrowed down our investigations on VEGFB because: (1) among the 4 pro angiogenic genes over-expressed in the high EF group (and thus potentially implicated in the favourable remodelling of the heart), only VEGFB was retrieved by the Entrez Gene database using the query: “angiogenesis AND homo sapiens AND heart”; and (2) the difference between VEGFB expression in high and low EF patients was the most significant among the pro angiogenic genes (Table 6).

Expression of VEGFB is Correlated with Outcome after MI

Quantitative PCR was used to confirm microarrays data on VEGFB. Expression values between the 2 groups of 16 MI patients were compared between microarrays and quantitative PCR. Both techniques reported higher levels of VEGFB mRNA expression in high EF patients compared with low EF patients: 1.3 fold (t=3.35; P=0.004) for microarrays, and 1.7 fold (t=3.35; P=0.003) for quantitative PCR (FIG. 8A). FIG. 8B displays the significant correlation observed between VEGFB expression and the EF (r=0.39; P=0.03). Therefore, VEGFB expression in blood cells appears to be correlated with outcome after MI.

Plasma Levels of VEGFB are Associated with Clinical Outcome after MI

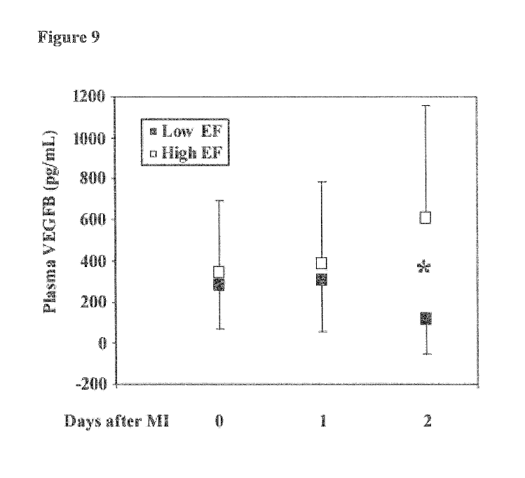

We then measured VEGFB in the plasma of 140 MI patients, separated in 2 groups, namely those with preserved (LVEF median 57%, range 45-89) and impaired (median 37%, range 17-44) LV function 1 month after MI. Blood sampling was performed the day of MI (n=77), 1 day after MI (n=65) or 2 days after MI (n=12). Plasma VEGFB levels were similar between high and low EF groups at day 0 and day 1 after MI. At day 2 however, VEGFB levels increased in high EF patients (2 fold compared with day 0) whereas they dropped in low EF patients (2.5 fold compared with day 0) (FIG. 9). These data are in accordance with the up regulation of VEGFB mRNA in high EF patients (FIG. 8). These results suggest that patients able to increase their production of VEGFB after MI are more prone to have a favourable outcome.

### Independent Validation

An independent cohort of 290 MI patients was used to further study the association between VEGFB plasma levels and LV remodelling post MI. Clinical characteristics of this patient population have been published. Mean plasma VEGFB was 64% higher (U Statistic=8128, P<0.001) in patients in whom AEDV (n=138) showed a fall over this period (n=138), compared with patients in whom AEDV increased (n=152). These data confirm our observation that VEGFB is associated with LV remodelling after MI.

### Prognostic Performance of VEGFB

Our results suggested that VEGFB could represent a potential biomarker of remodelling after MI. Two machine learning models built with several sets of data—obtained either by microarrays, quantitative PCR or plasma determination—were used to test the prognostic performance of VEGFB. Results are shown in Table 7. The best performance was achieved when the K* instance based learner was built with VEGFB expression levels measured in blood cells by microarrays from the 32 patients of the test cohort. This model reached a specificity of 75% (12 of 16 low EF patients correctly classified), a sensitivity of 50% (8 of 16 high EF patients correctly classified), and an overall accuracy of 62% (20 of 32 patients correctly classified). AUC was 0.75. When built with plasma VEGFB levels from the validation cohort (290 patients), the maximal prognostic significance provided an AUC of 0.52.

TABLE 7
Prediction performances of VEGFB.

| | | | Classi- | | Speci- | Sensi- | Accu- |
|---|---|---|---|---|---|---|---|
| | | | fication | | ficity | tivity | racy |
| | | n | model | AUC | (%) | (%) | (%) |
| VEGFB | Day 0 | 32 | SVM | 0.56 | 56 | 56 | 56 |
| (micro- | | | K* | 0.75 | 75 | 50 | 62 |
| arrays) | | | | | | | |
| VEGFB | Day 0 | 32 | SVM | 0.68 | 94 | 44 | 69 |
| (PCR) | | | K* | 0.68 | 56 | 56 | 56 |
| VEGFB | Day 0 | 77 | SVM | 0.5 | 0 | 1 | 66 |
| (plasma) | | | K* | 0.51 | 0 | 92 | 60 |
| | Day 1 | 65 | SVM | 0.5 | 0 | 1 | 66 |
| | | | K* | 0.47 | 0 | 87 | 57 |
| | Day 2 | 12 | SVM | 0.5 | 0 | 1 | 66 |
| | | | K* | 0.07 | 0 | 1 | 66 |

AUC: Area Under the Receiver Operating Characteristic (ROC) Curve.

Specificity indicates the percentage of correctly classified low EF patient; sensitivity indicates the percentage of correctly classified high EF patient; accuracy indicates the percentage of correctly classified high and low EF patients.

1. A method of identifying myocardially-infarcted patients having an increased risk of developing a heart condition, comprising:
assaying, post infarction, a body fluid sample from the patient for levels of Vascular Endothelial Growth Factor B (VEGFB), Thrombospondin-1 (THBS1) and/or Placental Growth Factor (PGF);
comparing the levels of VEGFB, THBS1 and/or PGF with a control; and
determining whether the patient has an increased risk of developing a heart condition, based on said comparison.

2. A method according to claim 1, wherein the control is a reference value obtainable from a population of infarcted patients with a range of clinical outcomes.

3. A method according to claim 1, wherein the control is a corresponding level of VEGFB, THBS1 and/or PGF from a further sample from said patient.

4. A method according to claim 3, wherein the further sample is an earlier sample from the patient and at least one of the following indicates an increased likelihood of said patient suffering from said heart condition:
lower levels of VEGFB in the assayed patient sample compared to the VEGFB earlier sample;
higher levels of THBS1 in the assayed patient sample compared to the THBS1 earlier sample; and/or
higher levels of PGF in the assayed patient sample compared to the PGF earlier sample.

5. A method according to claim 1, wherein the heart condition is myocardial infarction, acute coronary syndrome, ischemic cardiomyopathy or non-ischemic cardiomyopathy, or wherein the patient develops or suffers from heart failure or ventricular remodeling.

6. A method according to claim 1, wherein the body fluid sample taken from the patient is a blood sample, a tissue fluid sample, a plasma sample, a serum sample or a urine sample.

7. A method according to claim 1, wherein the levels of VEGFB, THBS1 and/or PGF assayed are mRNA levels or plasma protein levels.

8. A method according to claim 1, wherein the VEGFB, THBS1 and/or PGF levels are assayed the day of myocardial infarction.

9. A method according to claim 1, wherein the determination is by nearest neighbor statistical analysis comparison techniques.

10. A method according to claim 1, further comprising collecting data on one or more MI patients, including analysis of the levels or values of at least one of VEGFB, THBS1 and/or PGF and the associated clinical outcome for that patient to create feature data for VEGFB, THBS1 and/or PGF associated with a particular clinical outcome.

11. A method according to claim 1, wherein a classifier is used to determine the prognosis, the classifier including programs such as PAM, Kstar and SVM.

12. A method according to claim 1, wherein the body fluid sample is assayed for the level of VEGFB and compared to the VEGFB reference level and, optionally, the body fluid sample is assayed for the level of one or both of THBS1 or PGF and compared to the corresponding THBS1 or PGF reference level.

13. A method of identifying a myocardially-infarcted patient having an increased risk of developing a heart condition, comprising:
analyzing a body fluid sample from a post-MI patient for mRNA or plasma protein levels of VEGFB, THBS1 and/or PGF to determine feature data of the patient, associating this feature data with a particular clinical outcome relating to the incidence of Heart Failure in said patient, and entering this feature data into a database;
repeating the analysis for a plurality of post-MI patients, to populate the database to contain reference information about the relationship between HF and levels of VEGFB, THBS1 and/or PGF;
determining feature data of a post-MI patient with an unknown prognosis; and
processing the feature data for the VEGFB, THBS1 and/or PGF levels from the patient to compare it with the feature data in the database; and
outputting a likely prognosis for the patient in dependence upon the result of the comparison.

14. A method according to claim 1 or 13, wherein the determination of an increased risk of a heart condition is relative to those infarcted reference samples having comparatively high levels of VEGFB mRNA (log ratio>−1.4), relatively low levels of THBS1 (log ratio<0) and/or relatively low levels PGF (log ratio<−0.1), compared to the queried sample, respectively.

15. A method according to claim 14, wherein ratios values vary by an amount selected from the group consisting of: at least 1 or 2%, at least 5%, at least 7%, at least 10%, at least 15%, at least 20%, at least 25%, at least 30%, at least 40%, and up to 50%.

16. A method according to claim 1 or 13, further comprising assaying the level of BNP and comparing this to a BNP reference sample.

17. A method of establishing a prognosis for a myocardially-infarcted patient, the method comprising:
assaying, post infarction, a body fluid sample from the patient for levels of Vascular Endothelial Growth Factor B (VEGFB) and levels of Thrombospondin-1 (THBS1) and/or Placental Growth Factor (PGF); and
comparing the levels VEGFB, THBS1 and/or PGF with a control; and
determining the prognosis for said patient based on said comparison.

18. A method of establishing a prognosis for a myocardially-infarcted patient, the method comprising:
assaying, post infarction, a body fluid sample from the patient for levels of Vascular Endothelial Growth Factor B (VEGFB) and levels of Thrombospondin-1 (THBS1) and/or Placental Growth Factor (PGF); and
questioning a previously built statistical program (also called “classifier”) with the levels of VEGFB, THBS1 and PGF;
wherein
a high level of VEGFB and low levels of THBS1 and PGF are associated with decreased likelihood of developing said heart condition; and
the classifier will dictate whether the patient has an decreased likelihood of developing said heart condition.

19. A method of identifying myocardially-infarcted patients having a reduced risk of said heart condition, comprising:
assaying, post infarction, a body fluid sample from the patient for levels of Vascular Endothelial Growth Factor B (VEGFB) and levels of Thrombospondin-1 (THBS1) and/or Placental Growth Factor (PGF);
comparing the levels VEGFB, THBS1 and/or PGF with a control; and
determining whether the patient has a reduced risk of developing a heart condition, based on said comparison.

20. A method for assaying VEGFB levels in a sample, comprising:
(a) contacting the sample with at least one capture reagent immobilized to a support to form an immobilized capture reagent-sample complex;
(b) separating the sample from the at least one immobilized capture reagent;
(c) contacting the immobilized capture reagent-sample complex with a secondary antibody specific for VEGFB and optionally contacting the secondary antibody with a tertiary antibody specific for the secondary antibody;
(d) contacting the secondary or tertiary antibody with a binding molecule, such as streptavidin, conjugated to detection means; and
(e) measuring the level of the secondary or tertiary antibody bound to the capture reagents using the detection means.

21. An ELISA kit to measure levels of VEGFB 186 and VEGFB 167 in biological fluids comprising;
(a) at least one capture reagent immobilized to a support;
(b) a secondary antibody specific for VEGFB 186 and/or VEGFB 167;
(c) optionally, a tertiary antibody specific for the secondary antibody;
(d) a binding molecule, such as streptavidin, conjugated to detection means; and
(e) means for measuring the level of the secondary or tertiary antibody bound to the capture reagents using the detection means.

22. A database comprising feature data from MI patients, the data including the clinical outcome of the patient matched to at least one of VEGFB, THBS1 and PGF levels assayed post-MI.

23. A database as claimed in claim 22, wherein the clinical outcome of the patient is matched to VEGFB levels assayed post-MI.

24. A comparing device for accessing the database according to claim 22 or 23 and/or processing the query.

25. A system comprising a database according to claim 22 or 23 and at least one comparing device according to claim 24 to access and/or operate the database.


SEQUENCE LISTING (13 sequences)

SEQ ID NO: 1 - DNA, 1172 bp, Homo sapiens
gcgatgcgggcgcccccggcgggcggccccggcgggcaccatgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttagagctcaacccagacacctgcaggtgccggaagctgcgaaggtgacacatggcttttcagactcagcagggtgacttgcctcagaggctatatcccagtgggggaacaaagaggagcctggtaaaaaacagccaagcccccaagacctcagcccaggcagaagctgctctaggacctgggcctctcagagggctcttctgccatcccttgtctccctgaggccatcatcaaacaggacagagttggaagaggagactgggaggcagcaagaggggtcacataccagctcaggggagaatggagtactgtctcagtttctaaccactctgtgcaagtaagcatcttacaactggctcttcctcccctcactaagaagacccaaacctctgcataatgggatttgggctttggtacaagaactgtgacccccaaccctgataaaagagatggaaggaaaaaaaaaaaaaaaaaaaaaaaaaaaaaaaaaaaaaaaaaaaaaaaaaaaaaaa

SEQ ID NO: 2 - DNA, 624 bp, Homo sapiens
atgagccctctgctccgccgcctgctgctcgccgcactcctgcagctggcccccgcccaggcccctgtctcccagcctgatgcccctggccaccagaggaaagtggtgtcatggatagatgtgtatactcgcgctacctgccagccccgggaggtggtggtgcccttgactgtggagctcatgggcaccgtggccaaacagctggtgcccagctgcgtgactgtgcagcgctgtggtggctgctgccctgacgatggcctggagtgtgtgcccactgggcagcaccaagtccggatgcagatcctcatgatccggtacccgagcagtcagctgggggagatgtccctggaagaacacagccagtgtgaatgcagacctaaaaaaaaggacagtgctgtgaagccagacagggctgccactccccaccaccgtccccagccccgttctgttccgggctgggactctgcccccggagcaccctccccagctgacatcacccatcccactccagccccaggcccctctgcccacgctgcacccagcaccaccagcgccctgacccccggacctgccgctgccgctgccgacgccgcagcttcctccgttgccaagggcggggcttag

SEQ ID NO: 3 - protein, 207 aa, Homo sapiens
MSPLLRRLLLAALLQLAPAQAPVSQPDAPGHQRKVVSWIDVYTRATCQPREVVVPLTVELMGTVAKQLVPSCVTVQRCGGCCPDDGLECVPTGQHQVRMQILMIRYPSSQLGEMSLEEHSQCECRPKKKDSAVKPDRAATPHHRPQPRSVPGWDSAPGAPSPADITHPTPAPGPSAHAAPSTTSALTPGPAAAAADAAASSVAKGGA

SEQ ID NO: 4 - DNA, 5820 bp, Homo sapiens
agccgctgcgcccgagctggcctgcgagttcagggctcctgtcgctctccaggagcaacctctactccggacgcacaggcattccccgcgcccctccagccctcgccgccctcgccaccgctcccggccgccgcgctccggtacacacaggatccctgctgggcaccaacagctccaccatggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaatcatcaaattgttgattgaaagactgatcataaaccaatgctggtattgcaccttctggaactatgggcttgagaaaacccccaggatcacttctccttggcttccttcttttctgtgcttgcatcagtgtggactcctagaacgtgcgacctgcctcaagaaaatgcagttttcaaaaacagactcagcattcagcctccaatgaataagacatcttccaagcatataaacaattgctttggtttccttttgaaaaagcatctacttgcttcagttgggaaggtgcccattccactctgcctttgtcacagagcagggtgctattgtgaggccatctctgagcagtggactcaaaagcattttcaggcatgtcagagaagggaggactcactagaattagcaaacaaaaccaccctgacatcctccttcaggaacacggggagcagaggccaaagcactaaggggagggcgcatacccgagacgattgtatgaagaaaatatggaggaactgttacatgttcggtactaagtcattttcaggggattgaaagactattgctggatttcatgatgctgactggcgttagctgattaacccatgtaaataggcacttaaatagaagcaggaaagggagacaaagactggcttctggacttcctccctgatccccacccttactcatcacctgcagtggccagaattagggaatcagaatcaaaccagtgtaaggcagtgctggctgccattgcctggtcacattgaaattggtggcttcattctagatgtagcttgtgcagatgtagcaggaaaataggaaaacctaccatctcagtgagcaccagctgcctcccaaaggaggggcagccgtgcttatatttttatggttacaatggcacaaaattattatcaacctaactaaaacattccttttctcttttttcctgaattatcatggagttttctaattctctcttttggaatgtagattttttttaaatgctttacgatgtaaaatatttattttttacttattctggaagatctggctgaaggattattcatggaacaggaagaagcgtaaagactatccatgtcatctttgttgagagtcttcgtgactgtaagattgtaaatacagattatttattaactctgttctgcctggaaatttaggcttcatacggaaagtgtttgagagcaagtagttgacatttatcagcaaatctcttgcaagaacagcacaaggaaaatcagtctaataagctgctctgccccttgtgctcagagtggatgttatgggattctttttttctctgttttatcttttcaagtggaattagttggttatccatttgcaaatgttttaaattgcaaagaaagccatgaggtcttcaatactgttttaccccatcccttgtgcatatttccagggagaaggaaagcatatacacttttttctttcatttttccaaaagagaaaaaaatgacaaaaggtgaaacttacatacaaatattacctcatttgttgtgtgactgagtaaagaatttttggatcaagcggaaagagtttaagtgtctaacaaacttaaagctactgtagtacctaaaaagtcagtgttgtacatagcataaaaactctgcagagaagtattcccaataaggaaatagcattgaaatgttaaatacaatttctgaaagttatgttttttttctatcatctggtataccattgctttatttttataaattattttctcattgccattggaatagatatctcagattgtgtagatatgctatttaaataatttatcaggaaatactgcctgtagagttagtatttctatttttatataatgtttgcacactgaattgaagaattgttggttttttcttttttttgttttgtttttttttttttttttttttgcttttgacctcccatttttactatttgccaatacctttttctaggaatgtgcttttttttgtacacatttttatccattttacattctaaagcagtgtaagttgtatattactgtttcttatgtacaaggaacaacaataaatcatatggaaatttatatttataaaaaaaaaaaaaaaaaaaaaaaaaaaaaaa

SEQ ID NO: 5 - DNA, 3513 bp, Homo sapiens
atggggctggcctggggactaggcgtcctgttcctgatgcatgtgtgtggcaccaaccgcattccagagtctggcggagacaacagcgtgtttgacatctttgaactcaccggggccgcccgcaaggggtctgggcgccgactggtgaagggccccgacccttccagcccagctttccgcatcgaggatgccaacctgatcccccctgtgcctgatgacaagttccaagacctggtggatgctgtgcgggcagaaaagggtttcctccttctggcatccctgaggcagatgaagaagacccggggcacgctgctggccctggagcggaaagaccactctggccaggtcttcagcgtggtgtccaatggcaaggcgggcaccctggacctcagcctgaccgtccaaggaaagcagcacgtggtgtctgtggaagaagctctcctggcaaccggccagtggaagagcatcaccctgtttgtgcaggaagacagggcccagctgtacatcgactgtgaaaagatggagaatgctgagttggacgtccccatccaaagcgtcttcaccagagacctggccagcatcgccagactccgcatcgcaaaggggggcgtcaatgacaatttccagggggtgctgcagaatgtgaggtttgtctttggaaccacaccagaagacatcctcaggaacaaaggctgctccagctctaccagtgtcctcctcacccttgacaacaacgtggtgaatggttccagccctgccatccgcactaactacattggccacaagacaaaggacttgcaagccatctgcggcatctcctgtgatgagctgtccagcatggtcctggaactcaggggcctgcgcaccattgtgaccacgctgcaggacagcatccgcaaagtgactgaagagaacaaagagttggccaatgagctgaggcggcctcccctatgctatcacaacggagttcagtacagaaataacgaggaatggactgttgatagctgcactgagtgtcactgtcagaactcagttaccatctgcaaaaaggtgtcctgccccatcatgccctgctccaatgccacagttcctgatggagaatgctgtcctcgctgttggcccagcgactctgcggacgatggctggtctccatggtccgagtggacctcctgttctacgagctgtggcaatggaattcagcagcgcggccgctcctgcgatagcctcaacaaccgatgtgagggctcctcggtccagacacggacctgccacattcaggagtgtgacaagagatttaaacaggatggtggctggagccactggtccccgtggtcatcttgttctgtgacatgtggtgatggtgtgatcacaaggatccggctctgcaactctcccagcccccagatgaacgggaaaccctgtgaaggcgaagcgcgggagaccaaagcctgcaagaaagacgcctgccccatcaatggaggctggggtccttggtcaccatgggacatctgttctgtcacctgtggaggaggggtacagaaacgtagtcgtctctgcaacaaccccacaccccagtttggaggcaaggactgcgttggtgatgtaacagaaaaccagatctgcaacaagcaggactgtccaattgatggatgcctgtccaatccctgctttgccggcgtgaagtgtactagctaccctgatggcagctggaaatgtggtgcttgtccccctggttacagtggaaatggcatccagtgcacagatgttgatgagtgcaaagaagtgcctgatgcctgcttcaaccacaatggagagcaccggtgtgagaacacggaccccggctacaactgcctgccctgccccccacgcttcaccggctcacagcccttcggccagggtgtcgaacatgccacggccaacaaacaggtgtgcaagccccgtaacccctgcacggatgggacccacgactgcaacaagaacgccaagtgcaactacctgggccactatagcgaccccatgtaccgctgcgagtgcaagcctggctacgctggcaatggcatcatctgcggggaggacacagacctggatggctggcccaatgagaacctggtgtgcgtggccaatgcgacttaccactgcaaaaaggataattgccccaaccttcccaactcagggcaggaagactatgacaaggatggaattggtgatgcctgtgatgatgacgatgacaatgataaaattccagatgacagggacaactgtccattccattacaacccagctcagtatgactatgacagagatgatgtgggagaccgctgtgacaactgtccctacaaccacaacccagatcaggcagacacagacaacaatggggaaggagacgcctgtgctgcagacattgatggagacggtatcctcaatgaacgggacaactgccagtacgtctacaatgtggaccagagagacactgatatggatggggttggagatcagtgtgacaattgccccttggaacacaatccggatcagctggactctgactcagaccgcattggagatacctgtgacaacaatcaggatattgatgaagatggccaccagaacaatctggacaactgtccctatgtgcccaatgccaaccaggctgaccatgacaaagatggcaagggagatgcctgtgaccacgatgatgacaacgatggcattcctgatgacaaggacaactgcagactcgtgcccaatcccgaccagaaggactctgacggcgatggtcgaggtgatgcctgcaaagatgattttgaccatgacagtgtgccagacatcgatgacatctgtcctgagaatgttgacatcagtgagaccgatttccgccgattccagatgattcctctggaccccaaagggacatcccaaaatgaccctaactgggttgtacgccatcagggtaaagaactcgtccagactgtcaactgtgatcctggactcgctgtaggttatgatgagtttaatgctgtggacttcagtggcaccttcttcatcaacaccgaaagggacgatgactatgctggatttgtctttggctaccagtccagcagccgcttttatgttgtgatgtggaagcaagtcacccagtcctactgggacaccaaccccacgagggctcagggatactcgggcctttctgtgaaagttgtaaactccaccacagggcctggcgagcacctgcggaacgccctgtggcacacaggaaacacccctggccaggtgcgcaccctgtggcatgaccctcgtcacataggctggaaagatttcaccgcctacagatggcgtctcagccacaggccaaagacgggtttcattagagtggtgatgtatgaagggaagaaaatcatggctgactcaggacccatctatgataaaacctatgctggtggtagactagggttgtttgtcttctctcaagaaatggtgttcttctctgacctgaaatacgaatgtagagatccctaa

SEQ ID NO: 6 - protein, 1170 aa, Homo sapiens
MGLAWGLGVLFLMHVCGTNRIPESGGDNSVFDIFELTGAARKGSGRRLVKGPDPSSPAFRIEDANLIPPVPDDKFQDLVDAVRAEKGFLLLASLRQMKKTRGTLLALERKDHSGQVFSVVSNGKAGTLDLSLTVQGKQHVVSVEEALLATGQWKSITLFVQEDRAQLYIDCEKMENAELDVPIQSVFTRDLASIARLRIAKGGVNDNFQGVLQNVRFVFGTTPEDILRNKGCSSSTSVLLTLDNNVVNGSSPAIRTNYIGHKTKDLQAICGISCDELSSMVLELRGLRTIVTTLQDSIRKVTEENKELANELRRPPLCYHNGVQYRNNEEWTVDSCTECHCQNSVTICKKVSCPIMPCSNATVPDGECCPRCWPSDSADDGWSPWSEWTSCSTSCGNGIQQRGRSCDSLNNRCEGSSVQTRTCHIQECDKRFKQDGGWSHWSPWSSCSVTCGDGVITRIRLCNSPSPQMNGKPCEGEARETKACKKDACPINGGWGPWSPWDICSVTCGGGVQKRSRLCNNPTPQFGGKDCVGDVTENQICNKQDCPIDGCLSNPCFAGVKCTSYPDGSWKCGACPPGYSGNGIQCTDVDECKEVPDACFNHNGEHRCENTDPGYNCLPCPPRFTGSQPFGQGVEHATANKQVCKPRNPCTDGTHDCNKNAKCNYLGHYSDPMYRCECKPGYAGNGIICGEDTDLDGWPNENLVCVANATYHCKKDNCPNLPNSGQEDYDKDGIGDACDDDDDNDKIPDDRDNCPFHYNPAQYDYDRDDVGDRCDNCPYNHNPDQADTDNNGEGDACAADIDGDGILNERDNCQYVYNVDQRDTDMDGVGDQCDNCPLEHNPDQLDSDSDRIGDTCDNNQDIDEDGHQNNLDNCPYVPNANQADHDKDGKGDACDHDDDNDGIPDDKDNCRLVPNPDQKDSDGDGRGDACKDDFDHDSVPDIDDICPENVDISETDFRRFQMIPLDPKGTSQNDPNWVVRHQGKELVQTVNCDPGLAVGYDEFNAVDFSGTFFINTERDDDYAGFVFGYQSSSRFYVVMWKQVTQSYWDTNPTRAQGYSGLSVKVVNSTTGPGEHLRNALWHTGNTPGQVRTLWHDPRHIGWKDFTAYRWRLSHRPKTGFIRVVMYEGKKIMADSGPIYDKTYAGGRLGLFVFSQEMVFFSDLKYECRDP

SEQ ID NO: 7 - DNA, 1758 bp, Homo sapiens
ctgctgtctgcggaggaaactgcatcgacggacggccgcccagctacgggaggacctggagtggcactgggcgcccgacggaccatccccgggacccgcctgcccctcggcgccccgccccgccgggccgctccccgtcgggttccccagccacagccttacctacgggctcctgactccgcaaggcttccagaagatgctcgaaccaccggccggggcctcggggcagcagtgagggaggcgtccagccccccactcagctcttctcctcctgtgccaggggctccccgggggatgagcatggtggttttccctcggagccccctggctcgggacgtctgagaagatgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaacccaccccttggaggagagagaccccgcacccggctcgtgtatttattaccgtcacactcttcagtgactcctgctggtacctgccctctatttattagccaactgtttccctgctgaatgcctcgctcccttcaagacgaggggcagggaaggacaggaccctcaggaattcagtgccttcaacaacgtgagagaaagagagaagccagccacagacccctgggagcttccgctttgaaagaagcaagacacgtggcctcgtgaggggcaagctaggccccagaggccctggaggtctccaggggcctgcagaaggaaagaagggggccctgctacctgttcttgggcctcaggctctgcacagacaagcagcccttgctttcggagctcctgtccaaagtagggatgcggatcctgctggggccgccacggcctggctggtgggaaggccggcagcgggcggaggggatccagccacttccccctcttcttctgaagatcagaacattcagctctggagaacagtggttgcctgggggcttttgccactccttgtcccccgtgatctcccctcacactttgccatttgcttgtactgggacattgttctttccggccaaggtgccaccaccctgccccccctaagagacacatacagagtgggccccgggctggagaaagagctgcctggatgagaaacagctcagccagtggggatgaggtcaccaggggaggagcctgtgcgtcccagctgaaggcagtggcaggggagcaggttccccaagggccctggcacccccacaagctgtccctgcagggccatctgactgccaagccagattctcttgaataaagtattctagtgtggaaaaaaaaaaaaaaaaaaaaaaaaaaaaaa

SEQ ID NO: 8 - DNA, 513 bp, Homo sapiens
atgccggtcatgaggctgttcccttgcttcctgcagctcctggccgggctggcgctgcctgctgtgcccccccagcagtgggccttgtctgctgggaacggctcgtcagaggtggaagtggtacccttccaggaagtgtggggccgcagctactgccgggcgctggagaggctggtggacgtcgtgtccgagtaccccagcgaggtggagcacatgttcagcccatcctgtgtctccctgctgcgctgcaccggctgctgcggcgatgagaatctgcactgtgtgccggtggagacggccaatgtcaccatgcagctcctaaagatccgttctggggaccggccctcctacgtggagctgacgttctctcagcacgttcgctgcgaatgccggcctctgcgggagaagatgaagccggaaaggaggagacccaagggcagggggaagaggaggagagagaagcagagacccacagactgccacctgtgcggcgatgctgttccccggaggtaa

SEQ ID NO: 9 - protein, 170 aa, Homo sapiens
MPVMRLFPCFLQLLAGLALPAVPPQQWALSAGNGSSEVEVVPFQEVWGRSYCRALERLVDVVSEYPSEVEHMFSPSCVSLLRCTGCCGDENLHCVPVETANVTMQLLKIRSGDRPSYVELTFSQHVRCECRPLREKMKPERRRPKGRGKRRREKQRPTDCHLCGDAVPRR

SEQ ID NO: 10 - DNA, 708 bp, Homo sapiens
ccccgcaggctgagggcaggtgggaagcaaacccggacgcatcgcagcagcagcagcagcagcagaagcagcagcagcagcctccgcagtccctccagagacatggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaagaggaagtcctggctgcagacacctgcttctgattccacaaggggctttttcctcaaccctgtggccgcctttgaagtgactcatttttttaatgtatttatgtatttatttgattgttttatataagatggtttcttacctttgagcacaaaatttccacggtgaaataaagtcaacattataagctttaaaaaaaaaaa

SEQ ID NO: 11 - DNA, 405 bp, Homo sapiens
atggatccccagacagcaccttcccgggcgctcctgctcctgctcttcttgcatctggctttcctgggaggtcgttcccacccgctgggcagccccggttcagcctcggacttggaaacgtccgggttacaggagcagcgcaaccatttgcagggcaaactgtcggagctgcaggtggagcagacatccctggagcccctccaggagagcccccgtcccacaggtgtctggaagtcccgggaggtagccaccgagggcatccgtgggcaccgcaaaatggtcctctacaccctgcgggcaccacgaagccccaagatggtgcaagggtctggctgctttgggaggaagatggaccggatcagctcctccagtggcctgggctgcaaagtgctgaggcggcattaa

SEQ ID NO: 12 - protein, 134 aa, Homo sapiens
MDPQTAPSRALLLLLFLHLAFLGGRSHPLGSPGSASDLETSGLQEQRNHLQGKLSELQVEQTSLEPLQESPRPTGVWKSREVATEGIRGHRKMVLYTLRAPRSPKMVQGSGCFGRKMDRISSSSGLGCKVLRRH

SEQ ID NO: 13 - protein, 184 aa, Homo sapiens
FDAIAEIGNQLYLFKDGKYWRFSEGRGSRPQGPFLIADKWPALPRKLDSVFEERLSKKLFFFSGRQVWVYTGASVLGPRRLDKLGLGADVAQVTGALRSGRGKMLLFSGRRLWRFDVKAQMVDPRSASEVDRMFPGVPLDTHDVFQYREKAYFCQDRFYWRVSSRSELNQVDQVGYVTYDILQC